(19) United States
(12) Patent Application Publication
(10) Pub. No.: US 2010/0113390 A1
(43) Pub. Date: May 6, 2010

(54) OLIGOSACCHARIDES DERIVED FROM FUCOIDAN

(75) Inventors: Shigeaki Fujikawa, Osaka (JP); Yuko Fukui, Osaka (JP); Hiroshi Watanabe, Osaka (JP); Yuji Nonaka, Kanagawa (JP); Takeshi Yasumoto, Miyagi (JP); Hideo Naoki, Osaka (JP)

Correspondence Address: DRINKER BIDDLE & REATH (DC), 1500 K STREET, N.W., SUITE 1100, WASHINGTON, DC, 20005-1209, US

(73) Assignees: SUNTORY HOLDINGS LIMITED, Osaka (JP); TROPICAL TECHNOLOGY CENTER LTD., Okinawa (JP)

(21) Appl. No.: 12/524,414

(22) PCT Filed: Jan. 26, 2007
(86) PCT No.: PCT/JP2007/051314

§ 371 Date: Jul. 24, 2009

(51) Int. Cl. A61K 31/715; C07H 3/04; C07H 3/06; C07H 5/10; A61P 3/04

(52) U.S. Cl. 514/61; 536/123.1; 536/123.13; 514/53; 514/54

(57) ABSTRACT

The present invention provides a fucoidan-derived low molecular weight compound with a good quality of taste, which has a specified structure and function and is free from problems in absorption, antigenicity, uniformity, an anticoagulant activity and so on, which problems arise when developing fucoidan, a sulfated polysaccharide having an extremely large molecular weight, as drugs or health foods. As a result of analyzing low molecular weight compounds obtained by acid hydrolysis of fucoidan, the inventors have identified fucoidan oligosaccharides (I) to (XI). Further, these oligosaccharides have been found to have anti-obesity and/or blood glucose elevation suppressing effects through inhibition of carbohydrate and/or lipid absorption as a result of α-glucosidase inhibition and/or lipase inhibition.

### TECHNICAL FIELD

The present invention relates to a new compound or a composition containing the compound, which has α-glucosidase and lipase inhibitory activity and can be utilized in foods and beverages, health food, physiologically functional food, medicines, cosmetics, etc. that aim to prevent obesity and hyperglycemia through inhibition of carbohydrate and/or lipid absorption.

### BACKGROUND ART

It has been reported that fucoidan, which is a sulfated polysaccharide contained in algae, has various activities including anticoagulant, lipemia-clearing (effect for removing cholesterol and lipoperoxide from blood), antitumor, cancer metastasis inhibitory and anti-AIDS virus infection effects.

It is known that the structure of fucoidan differs depending on an alga from which the fucoidan is originated, its growth environment, etc. One of the reasons is that compositions of fucose, galactose, xylose, glucuronic acid, and the like, which are components of fucoidan, vary depending on algae and their growth environment. Furthermore, positions of an ester bond and a glucoside bond on the constituent sugars may vary, contributing to diversity of the structure of fucoidan. Therefore, structures of many types of fucoidans have not been identified. Moreover, fucoidan has an offensive taste originating from source material, which limits the use of fucoidan in food.

Because of these reasons, when developing foods and beverages, medicines, and so on, by utilizing fucoidan, a lot of time has been needed in order to select fucoidan suitable for them. Also consumers do not know exactly of which fucoidan should be selected. Moreover, since fucoidan is a sulfated polysaccharide having an extremely large molecular weight, there are problems in absorption, antigenicity, uniformity, anticoagulant activity and so on, when fucoidan itself is used in foods and beverages or medicines.

Until now, chemically synthesized oligosaccharides containing fucose have been reported (Non-Patent Documents 1, 2, and 3).

A method for reducing the molecular weight of fucoidan by hydrolysis has been reported. For example, Patent Document 1 discloses a method of acid hydrolysis of fucoidan and describes that the resulting fucoidan with a low molecular weight had a molecular weight distribution of 5×10³ or lower. Patent Document 2 describes a method for producing an oligosaccharide by hydrolysis of fucoidan without adding an acid from outside. As in Patent Document 3, a method of hydrolysis of fucoidan by an enzyme has been also reported.

Some oligosaccharides have been reported, which oligosaccharides are obtained by hydrolysis of fucoidan, and their structures are determined. For example, Patent Document 4 reports that an oligosaccharide was produced by acid hydrolysis of fucoidan that was obtained from algae such as Nemacystus decipiens, and specifies the structures of several types of low molecular weight oligosaccharides derived from fucoidan. Patent Documents 5 and 6 disclose the structures of oligosaccharides obtained by enzymatic hydrolysis of fucoidan.

Further, Non-patent Document 4 shows the presence of GF and the presence of oligosaccharides having 1 or 2 fucose molecules which are sulfated partially.

Moreover, chondroitin sulfate and chitosan are known to have lipase inhibitory activity (Non-patent Documents 5 and 6).

Patent Document 1: Japanese Patent Laid-Open No. H7-215990

Patent Document 2: Japanese Patent Laid-Open No. 2002-226496

Patent Document 3: Japanese Patent Laid-Open No. 2000-236889

Patent Document 4: Japanese Patent Laid-Open No. 2000-351790

Patent Document 5: Japanese Patent Laid-Open No. 2003-199596

Patent Document 6: Japanese Patent Laid-Open No. 2001-226408

Patent Document 7: Japanese Patent Laid-Open No. H6-65080

Patent Document 8: Japanese Patent Laid-Open No. H8-23973

Patent Document 9: Japanese Patent Laid-Open No. H10-290681

Non-patent Document 1: Carbohydrate research 4, 189-195 (1967)

Non-patent Document 2: Carbohydrate research 37, 75-79 (1974)

Non-patent Document 3: Carbohydrate research 41, 308-312 (1975)

Non-patent Document 4: Glycoconjugate Journal 16, 19-26 (1999)

Non-patent Document 5: International Journal of obesity 24, 1131-1138 (2000)

Non-patent Document 6: International Journal of obesity 23, 174-179 (1999)

Non-patent Document 7: Infection and Immunity, 35, 71-78 (1982)

### DISCLOSURE OF THE INVENTION

### Problems to be Solved by the Invention

However, the structures of the substances obtained in the methods described in Patent Documents 1 to 3 have not been determined. Accordingly, there is a problem that quality management is not easy when using these oligosaccharides with unknown structures in food. Moreover, the substances obtained in the methods described in Patent Documents 1 to 3 were not preferred for use in food or the like, because they were prepared by organic synthesis reactions.

The functions of the oligosaccharide described in Patent Document 4 in food or the like are unclarified, and therefore it is difficult to say that its safety is high. Furthermore, the oligosaccharide described in Patent Document 5 has a problem that its molecular weight is large.

Further, Non-patent Document 4 shows the presence of GF and the presence of oligosaccharides having 1 or 2 fucose molecules which are sulfated partially, but no attempt has been made to isolate such substances and evaluate their properties.

Moreover, it is known that α-glucosidase inhibitory activity and lipase inhibitory activity are effective for an anti-obesity effect and/or an effect of suppressing blood glucose elevation (see Patent Documents 7, 8 and 9, and Non-patent Documents 5 and 6). As an example of oligosaccharides having α-glucosidase or lipase inhibitory activity, xylobiose has an α-glucosidase inhibitory effect, but it is too slow to obtain the effect. Further, there is no oligosaccharide known to have both α-glucosidase inhibitory activity and lipase inhibitory activity.

Therefore, as a material capable of being used in various applications, it is desired to develop an oligosaccharide derived from fucoidan, which has a specified structure and is capable of being quality-managed precisely, as described above. In view of applications in foods and beverages, and medicines, it is necessary to have a small molecular weight, to be easy to handle, and furthermore to have high safety, and also to have no offensive taste.

### Means for Solving the Problems

Therefore, an object of the present invention is to provide a new oligosaccharide derived from fucoidan having a specified structure.

Another object of the present invention is to provide an oligosaccharide derived from fucoidan that is highly safe, has α-glucosidase inhibitory activity and/or lipase inhibitory activity, has an anti-obesity effect and/or an effect of suppressing blood glucose elevation (blood glucose elevation suppressing effect) through inhibition of carbohydrate and/or lipid absorption, and further has no offensive taste and has a good quality of taste. A further object of the present invention is to provide an oligosaccharide derived from fucoidan, of which effective amount can be appropriately added to foods and beverages, pharmaceutical compositions, cosmetics, etc.

The present inventors have produced new oligosaccharides from fucoidan, and confirmed their α-glucosidase inhibitory activity and/or lipase inhibitory activity, and further confirmed their quality of taste, to complete the present invention. In the present invention, these oligosaccharides are also referred to as a fucoidan oligosaccharide.

That is, the present invention relates to:

(1) a fucoidan oligosaccharide represented by the following structural formula (I), (II), (III), (IV), (V), (VI), (VII), (VIII), (IX), (X), (XI) or (XII):

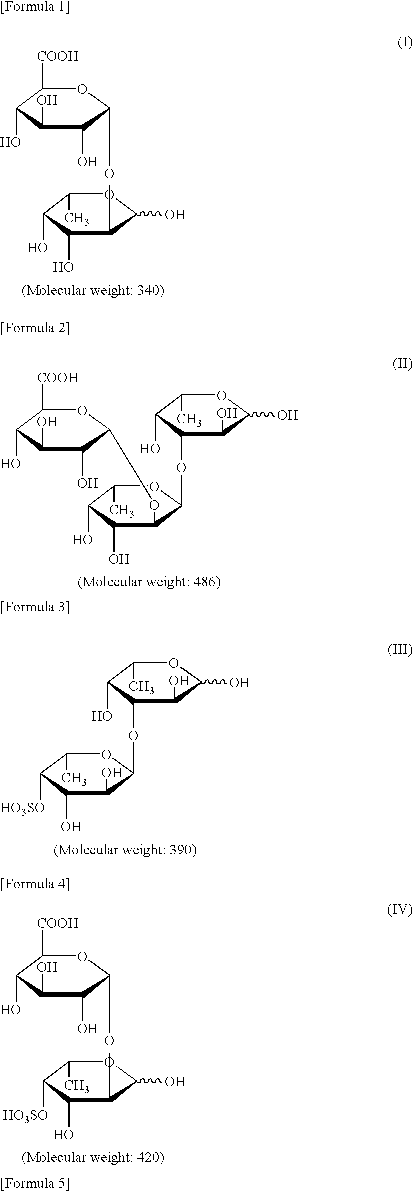

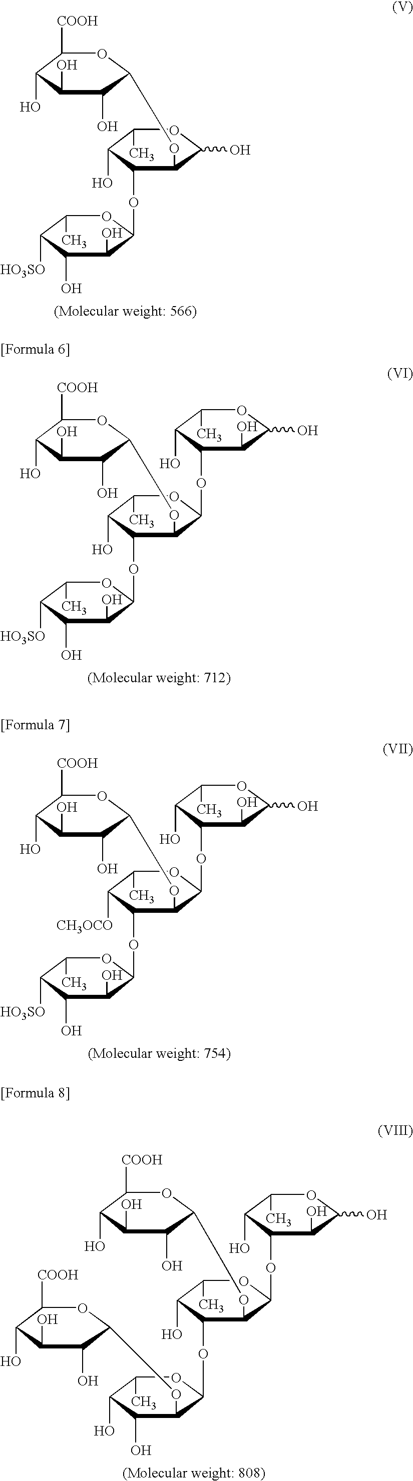

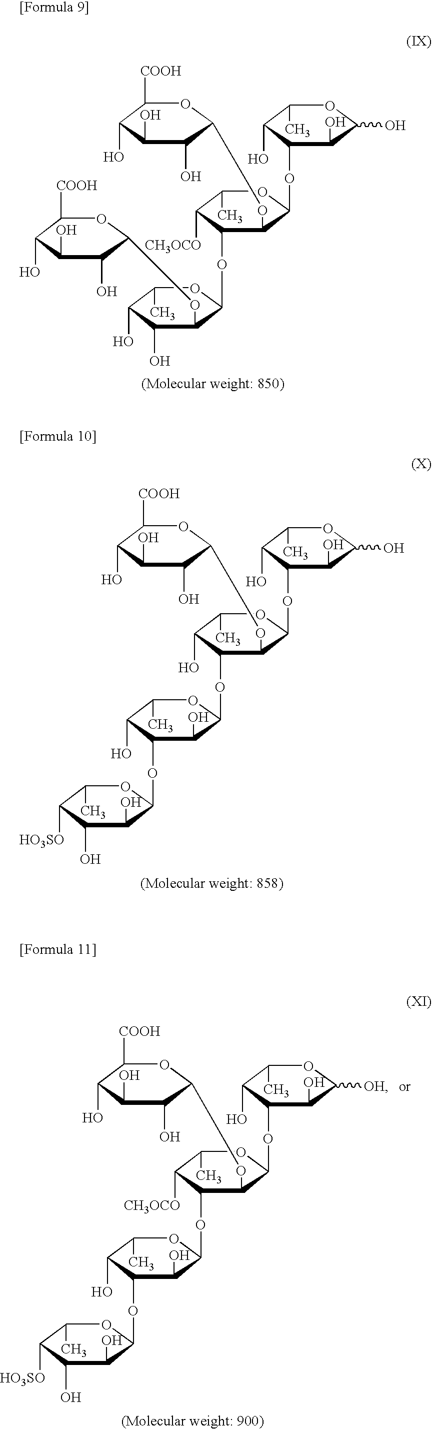

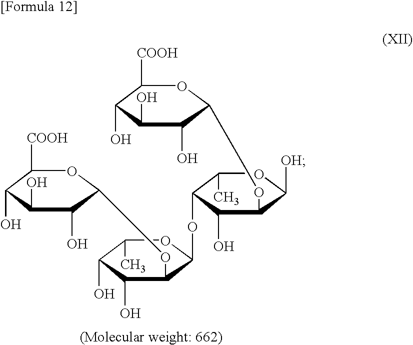

(2) an α-glucosidase inhibitor or a lipase inhibitor, which comprises at least one compound selected from compounds represented by formulae (I) to (XII);
(3) an anti-obesity agent or a blood glucose elevation suppressing agent, which comprises at least one compound selected from compounds represented by formulae (I) to (XII);
(4) a food or beverage, which incorporates at least one compound selected from compounds represented by formulae (I) to (XII); and
(5) a cosmetic, which comprises at least one compound selected from compounds represented by formulae (I) to (XII).

### ADVANTAGES OF THE INVENTION

New fucoidan oligosaccharides of the present invention have α-glucosidase and/or lipase inhibitory effects. The fucoidan oligosaccharides of the present invention have very high safety and a good taste, because they are separated from a food material. Therefore, the oligosaccharides of the present invention are very useful, and can be applied not only in health food, but also in medicines and cosmetics.

That is, addition of the oligosaccharides of the present invention can provide foods and beverages, pharmaceutical compositions, or cosmetics that have anti-obesity and/or blood glucose elevation suppressing effects due to the ability of the oligosaccharides to inhibit carbohydrate and/or lipid absorption.

### BRIEF DESCRIPTION OF THE DRAWINGS

FIG. 1 is an HPLC chart showing sugar composition analysis of fucoidan obtained by hot water extraction of Okinawa Nemacystus decipiens.

FIG. 2 shows an MS spectrum of a fucoidan oligosaccharide having a molecular weight of 340 represented by formula (I).

FIG. 3 shows an MS spectrum of a fucoidan oligosaccharide having a molecular weight of 486 represented by formula (II).

FIG. 4 shows a ¹H-NMR spectrum of a labeled oligosaccharide corresponding to the compound of formula (I).

FIG. 5 shows a ¹³C-NMR spectrum of a labeled oligosaccharide corresponding to the compound of formula (I).

FIG. 6 shows a ¹H-NMR spectrum of a labeled oligosaccharide corresponding to the compound of formula (II).

FIG. 7 shows a ¹³C-NMR spectrum of a labeled oligosaccharide corresponding to the compound of formula (II).

FIG. 8 shows a ¹H-NMR spectrum of a labeled oligosaccharide having a molecular weight of 539 corresponding to the compound of formula (III).

FIG. 9 shows a ¹³C-NMR spectrum of a labeled oligosaccharide having a molecular weight of 539 corresponding to the compound of formula (III).

FIG. 10 shows a ¹H-NMR spectrum of a labeled oligosaccharide having a molecular weight of 715 corresponding to the compound of formula (V).

FIG. 11 shows a ¹³C-NMR spectrum of a labeled oligosaccharide having a molecular weight of 715 corresponding to the compound of formula (V).

FIG. 12 shows a ¹H-NMR spectrum of a labeled oligosaccharide having a molecular weight of 861 corresponding to the compound of formula (VI).

FIG. 13 shows a ¹³C-NMR spectrum of a labeled oligosaccharide having a molecular weight of 861 corresponding to the compound of formula (VI).

FIG. 14 shows a ¹H-NMR spectrum of a labeled oligosaccharide having a molecular weight of 903 corresponding to the compound of formula (VII).

FIG. 15 shows a ¹³C-NMR spectrum of a labeled oligosaccharide having a molecular weight of 903 corresponding to the compound of formula (VII).

FIG. 16 shows a ¹H-NMR spectrum of a labeled oligosaccharide having a molecular weight of 957 corresponding to the compound of formula (VIII).

FIG. 17 shows a ¹³C-NMR spectrum of a labeled oligosaccharide having a molecular weight of 957 corresponding to the compound of formula (VIII).

FIG. 18 shows a ¹H-NMR spectrum of a labeled oligosaccharide having a molecular weight of 999 corresponding to the compound of formula (IX).

FIG. 19 shows a ¹³C-NMR spectrum of a labeled oligosaccharide having a molecular weight of 999 corresponding to the compound of formula (IX).

FIG. 20 shows a ¹H-NMR spectrum of a fucoidan oligosaccharide having a molecular weight of 754 represented by formula (VII).

FIG. 21 shows a TOF-MS spectrum of a fucoidan oligosaccharide having a molecular weight of 754 represented by formula (VII).

FIG. 22 shows an MS/MS spectrum after reproducing a fucoidan oligosaccharide having a molecular weight of 754 represented by formula (VII).

FIG. 23 shows an ESI-MS spectrum of a fucoidan oligosaccharide having a molecular weight of 420 represented by formula (IV).

FIG. 24 shows an MS/MS spectrum of a fucoidan oligosaccharide having a molecular weight of 420 represented by formula (IV).

FIG. 25 shows FAB-MS spectra of a fucoidan oligosaccharide having a molecular weight of 858 represented by formula (X) and a fucoidan oligosaccharide having a molecular weight of 900 represented by formula (XI).

FIG. 26 shows an MS/MS spectrum of a fucoidan oligosaccharide having a molecular weight of 858 represented by formula (X).

FIG. 27 shows an MS/MS spectrum of a fucoidan oligosaccharide having a molecular weight of 900 represented by formula (XI).

FIG. 28 is an ESI-MS chart of a Okinawa Nemacystus decipiens hydrolysate which is fluorescently labeled by ABEE.

FIG. 29 shows the α-glucosidase inhibitory effect of various fucoidan oligosaccharides.

FIG. 30 shows the lipase inhibitory effect of various fucoidan oligosaccharides.

### BEST MODE FOR CARRYING OUT THE INVENTION

### Fucoidan

Fucoidan generally refers to sulfated polysaccharides originated from algae, and contains galactose, glucuronic acid, sulfated fucose, xylose, and so on in addition to a main constituent sugar, fucose. Kinds and amounts of constituent sugars defer depending on algae from which fucoidan is derived, and growth environments for the algae.

Fucoidan to be used as a raw material for fucoidan oligosaccharides of the present invention may have any structures, and may be obtained from any alga. Examples of the algae include seaweeds of the class Phaeophyceae comprising various orders such as Sphacelariales, Chordariales, Scytosiphonales, Dictyosiphonales, Cutleriales, Sporochnales, Dictyotales, Laminariales, and Fucales. Preferably, fucoidan derived from Nemacystus decipiens may be used and fucoidan derived from Okinawa Nemacystus decipiens is more preferred.

Method for Extracting Fucoidan

Various methods for extracting fucoidan from algae have been studied and widely known (for example, a method using water as described in Japanese Patent Laid-Open No. 10-245334, a method using an acid as described in Japanese Patent Laid-Open No. 10-195106, and a method using an aqueous alkaline solvent as described in Japanese Patent Laid-Open No. 2002-262788). Fucoidan that is to be used as a raw material of oligosaccharides of the present invention can be obtained by these known methods. For example, the present invention uses fucoidan obtained by the following method.

That is, to algae (for example, Okinawa Nemacystus decipiens), a 5 to 10-fold amount of distilled water is added, and conduct extraction at 50° C. to 100° C. for 0 to 5 hours, preferably at 80° C. to 100° C. for 0.5 to 2 hours, and more preferably at 90° C. to 100° C. for about 1 hour. The thus obtained algae extract can be cooled, filtered by suction, desalinated, and dried to obtain fucoidan fractions that are easily dissolved in water. Thus obtained fucoidan fractions may be used in a next step without further purification, or after further purification.

Fucoidan to be used in the present invention is preferably an algal extract that is obtained in the manner described above; if desired, it may be used in a form as is naturally contained in algae. Fucoidan or an alga that contains it is then subjected to the subsequent hydrolyzing step to give a compound of the present invention.

Method for Producing Fucoidan Oligosaccharide Mixture

In order to produce a fucoidan oligosaccharide of the present invention, at first, a mixture of fucoidan oligosaccharides is obtained by hydrolyzing fucoidan by a method using an acid or an enzyme as described in Patent Documents 1 to 3. Preferably, acid hydrolysis conditions as described below are used.

That is, a fucoidan-containing fraction or fucoidan obtained from algae as described above is decomposed using an acid, preferably hydrochloric acid or sulfuric acid. More specifically, hydrolysis is conducted in an aqueous solvent containing 0.1 to 5.0 N, preferably 0.5 to 4.0 N, more preferably 0.5 to 3.0 N HCl at 25° C. to 130° C., preferably 30° C. to 105° C., and more preferably 50° C. to 100° C. for 0.1 to 6 hours, preferably 0.25 to 3 hours, and more preferably 0.5 to 2 hours. A mixture of fucoidan oligosaccharides can be obtained by neutralizing the obtained reaction product with a base, for example, about 1 N NaOH, followed by desalination by appropriate means such as electrodialysis or gel filtration, and drying (for example, lyophilization).

Purification of Oligosaccharide

The fucoidan oligosaccharide mixture thus obtained may be treated with activated carbon or by desalination to remove impurities, thereby obtaining a highly purified fucoidan oligosaccharide mixture. In order to further purify a fucoidan oligosaccharide, a method such as chromatography, recrystallization, dialysis, and alcohol precipitation can be employed alone or in combination. For example, an oligosaccharide is purified in accordance with the following procedures.

At first, the oligosaccharide mixture obtained by hydrolysis of fucoidan is subjected to chromatography using an anion exchange resin and then separated into a fraction which simply passes through the column without being adsorbed and contains oligosaccharides free from sulfate groups (fraction of a neutral sugar and a glucuronic acid sugar), and a fraction which is eluted with an acidic eluent and contains oligosaccharides rich in sulfate groups (sulfated sugar fraction).

Alternatively, the oligosaccharide mixture obtained by hydrolysis of fucoidan may be subjected to chromatography using a weakly basic anion exchange resin and then separated into a fraction which simply passes through the column without being adsorbed (fraction of a neutral sugar such as fucose), a fraction which is eluted with a weak acid and contains oligosaccharides free from sulfate groups (glucuronic acid sugar fraction), and a fraction which is eluted with a strongly acidic eluent and contains oligosaccharides rich in sulfate groups (sulfated sugar fraction).

The latter half of the fraction eluted with a strongly acidic eluent may further be fractionated to obtain high-purity sulfated fucose which is free from sulfate group-containing oligosaccharides.

A disaccharide represented by formula (I) and a trisaccharide represented by formula (II) can be obtained by subjecting the fraction of neutral and glucuronic acid sugars to gel filtration.

Moreover, when the glucuronic acid sugar fraction is further subjected to chromatography such as preparative HPLC, it is possible to obtain a disaccharide represented by formula (I), a trisaccharide represented by formula (II), a tetrasaccharide represented by formula (XII) and a pentasaccharide represented by formula (VIII).

On the other hand, each component represented by formula (V), (VI) or (VII) can be isolated by subjecting the sulfated sugar fraction to chromatography such as preparative HPLC.

In order to facilitate purification and structural analysis, each of the oligosaccharides may be appropriately labeled or derivatized. For example, an oligosaccharide can be fluorescently labeled by use of a reagent such as ethyl 4-aminobenzoate (ABEE), thereby making it easy to detect the oligosaccharide. A pure oligosaccharide can be obtained if each of the labeled oligosaccharides is separated, and then the labeled part is removed.

Thus obtained fucoidan oligosaccharides not only may each be used alone, but also may be used as an oligosaccharide mixture obtained by removing impurities from the fucoidan hydrolysate with activated carbon or through electrodialysis, as an oligosaccharide mixture obtained by removing impurities from the fraction (glucuronic acid sugar fraction) which is obtained by ion exchange resin fractionation or the like and contains oligosaccharides almost free from sulfate groups, or as an oligosaccharide mixture obtained by removing impurities from the fraction (sulfated sugar fraction) which contains oligosaccharides rich in sulfate groups. These oligosaccharides or oligosaccharide mixtures can be used in, for example, foods and beverages, medicines, and cosmetics, allowing them to have α-glucosidase and/or lipase inhibitory effects, as well as anti-obesity and/or blood glucose elevation suppressing effects through inhibition of carbohydrate and/or lipid absorption.

Food Additive, and Foods and Beverages Comprising Fucoidan Oligosaccharide, as Well as Foods and Beverages Incorporating Fucoidan Oligosaccharide Added Thereto

When using the fucoidan oligosaccharide of the present invention in foods and beverages, the foods and beverages are suitably formed as the food additives and foods and beverages that contain the fucoidan oligosaccharide and have α-glucosidase and/or lipase inhibitory effects, as well as anti-obesity and/or blood glucose elevation suppressing effects through inhibition of carbohydrate and/or lipid absorption, and as health food that contains the fucoidan oligosaccharide added thereto and has α-glucosidase and/or lipase inhibitory effects, as well as anti-obesity and/or blood glucose elevation suppressing effects through inhibition of carbohydrate and/or lipid absorption.

They may be a product adapted to users' tastes by being mixed with various components such as known sweeteners, acidifiers, and vitamins. The foods and beverages can be provided in a form of, for example, tablets; capsules; refreshing beverages; tea beverages; drinks; dairy products such as yoghurts and lactic acid bacteria beverages; seasonings; processed food; desserts; and confectionery such as gum, candy, and jelly. The foods and beverages according to the present invention include physiologically functional foods (including FOSHUs, or foods for specified health use, and qualified FOSHUs) with an indication that states that they have α-glucosidase and/or lipase inhibitory effects to thereby produce anti-obesity and/or blood glucose elevation suppressing effects through inhibition of carbohydrate and/or lipid absorption, either on the container or in an instruction. The indication may be written on the container, written in a direction attached to the container, etc, but the place on which the indication is written is not limited thereto. The containers include bottles, cans, PET bottles, plastic bottles, and cartons, but not limited thereto. The indication methods include printing, stamping, and seals, but not limited thereto. The foods and beverages may be pet food processed as feed for pets or animal feed.

Pharmaceutical Composition Comprising Fucoidan Oligosaccharide

The fucoidan oligosaccharides of the present invention can be used as anti-obesity and/or blood glucose elevation suppressing agents having α-glucosidase and/or lipase inhibitory activity and exerting their efficacy through inhibition of carbohydrate and/or lipid absorption. Accordingly, in one aspect, the present invention is a pharmaceutical composition that comprises the fucoidan oligosaccharide of the present invention and has α-glucosidase and/or lipase inhibitory activity, as well as anti-obesity and/or blood glucose elevation suppressing effects through inhibition of carbohydrate and/or lipid absorption.

The pharmaceutical composition can be formulated adding known auxiliaries usually used in the field of pharmaceutical formulation technology such as diluents, carriers, binders, disintegrators, lubricants, flavoring agents, solubilizing agents, suspending agents, and coating agents to the active ingredient. Examples of the dosage forms include tablets, capsules, granules, powders, liquids, syrups, suppositories, creams, ointments, emulsion, adhesive skin patches, and injections, but not particularly limited thereto. Examples of the administration routes of the present pharmaceuticals include oral administration, rectal administration, and enteral administration, but not particularly limited thereto.

Cosmetic Comprising Fucoidan Oligosaccharide

By using the fucoidan oligosaccharide of the present invention, it is possible to produce a cosmetic having a lipase inhibitory effect to suppress the proliferation of acne bacteria through inhibition of lipid degradation (Non-patent Document 7), and thus having a prophylactic effect for skin disease such as comedo.

The cosmetics which the oligosaccharide of the present invention are added to or are mixed in are, for example, creams, lotions, gels, mousse, shampoo, and rinse for face, skin, and hair.

Combined Use with Other Components

The fucoidan oligosaccharide of the present invention may be used alone in foods and beverages, pharmaceutical compositions, and cosmetics, but it is also suitable to use the saccharide in combination with other food materials or substances that have α-glucosidase and/or lipase inhibitory effects to thereby produce anti-obesity and/or blood glucose elevation suppressing effects through inhibition of carbohydrate and/or lipid absorption. Examples of such food materials or substances include lactic acid bacteria, mushrooms, fucoidan, xylooligosaccharide, arabinose, xylose, and fucose.

Other than these active components, commonly used components such as carriers, diluents, excipients, or additives can be formulated into foods and beverages and compositions of the present invention, depending on specific aspects. Here, the carriers, diluents, or excipients are not particularly limited as long as they do not inhibit the physiological activity of the fucoidan oligosaccharides. The examples include sugars such as sucrose, glucose, arabinose, fructose, maltose, trehalose, lactose, starch, starch syrup, and high fructose syrup; alcohols such as ethanol, propylene glycol, and glycerin; sugar alcohols such as sorbitol, mannitol, erithritol, lactitol, xylitol, maltitol, reduced palatinose, and decomposed reduced starch; solvents such as triacetin; polysaccharides such as gum arabic, carrageenan, xanthan gum, guar gum, gellan gum, and pectin; or water. Examples of the additives include auxiliaries such as chelating agents; flavors; spice extracts, and antiseptic agents. Such carriers, additives, and the like can be added as long as they do not impair the effect of the present invention.

An amount of the oligosaccharides formulated in foods and beverages, pharmaceutical compositions, and cosmetics is appropriately selected depending on relationships with other selected formulation components and so on, not particularly limited. However, when the fucoidan oligosaccharide is added to beverage or food, or pharmaceutical compositions, the amount is usually 0.01 g to 10 g/day, preferably 0.05 g to 1 g/day, and particularly preferably 0.05 g to 0.5 g/day per 60 kg of body weight of an individual. In cosmetics, 0.01% to 20% by weight, and preferably 0.05% to 15% by weight is used.

An extracted purified product and a synthetic product of the oligosaccharide of the present invention can be used alone in foods and beverages, pharmaceutical compositions, and cosmetics, but the oligosaccharide can be added to foods and beverages, etc. in a form of a mixture of one or more oligosaccharides of the present invention.

The present invention is not limited to each of the aforementioned embodiments, but can be modified in various ways within the scope shown in the claims. Embodiments obtained by appropriately combining technical means each disclosed in different embodiments are also within the technical scope of the present invention.

### EXAMPLES

The present invention will be specifically described based on Examples below, but needless to say, the scope of the present invention is not limited to these Examples.

In the following Examples, unless particularly indicated, NMR analysis was carried out using ECA-600 type nuclear magnetic resonance apparatus (JEOL Ltd.). Deuterated water (D₂O) was used as a measurement solvent. Binding modes of constituent sugars were determined by 2D-NMR.

### Example 1

### Preparation of Fucoidan Oligosaccharide-1

### a) Preparation of Fucoidan Fraction

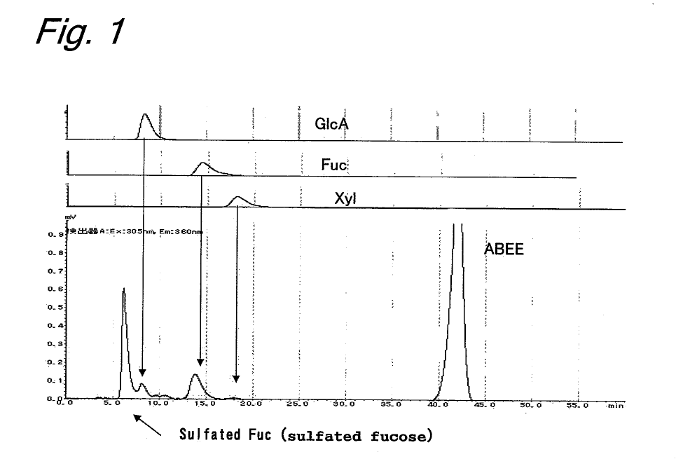

To 100 g of Okinawa Nemacystus decipiens, 1000 ml of distilled water was added, and extraction was conducted at 100° C. for 1 hour. The obtained extract was cooled, and then filtered by suction, electrodialyzed (desalinated), and lyophilized to obtain 2 g of fucoidan fractions. This fucoidan was hydrolyzed with an aqueous solution containing 2NH₂SO₄ at 100° C. for 1 hour. The obtained aqueous solution was neutralized by 2 N NaOH, and fluorescently labeled by ABEE to prepare a monosaccharide analysis sample. It was confirmed that the composition of the constituent sugars was sulfated fucose:glucuronic acid:fucose:xylose=49.3:4.9:12.1:1 (FIG. 1).

Column: Cosmosil C18 AR-II (4.6 mmφ×250 mm)
Mobile phase: 0.2 M potassium borate buffer containing 10% acetonitrile
Flow rate: 1.0 ml/min.

### Temperature: 45° C.

Detection: fluorescence detector (Shimadzu Corporation), Ex: 305 nm, Em: 360 nm

### b) Hydrolysis of Fucoidan and Separation of Oligosaccharides

To 1 g of the obtained fucoidan fractions, 100 ml of 2 N HCl was added, followed by acid hydrolysis at 50° C. to 100° C. for 1 hour and subsequent neutralization by 1 N NaOH. The obtained reaction solution was subjected to gel filtration (Biogel P-6 (Bio-Rad)) for desalination and then lyophilized to obtain 895 mg of a fucoidan oligosaccharide mixture. The obtained fucoidan oligosaccharide mixture was subjected to chromatography using an anion exchange resin activated by formate (TOSOH CORPORATION). As a result, the oligosaccharide mixture was separated into 280 mg of a fraction containing oligosaccharides free from sulfate groups obtained by elution with water (fraction of neutral and acidic sugars), and 425 mg of a fraction containing oligosaccharides rich in sulfate groups obtained by elution with 2 N HCl (sulfated sugar fraction).

### c) Isolation of Compounds (I) and (II)

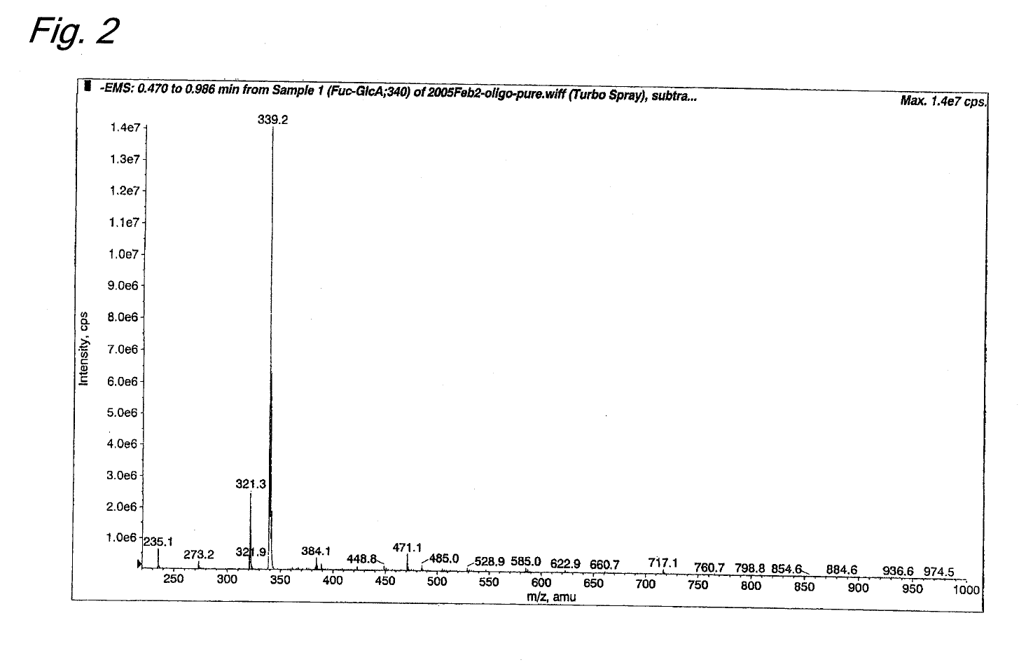

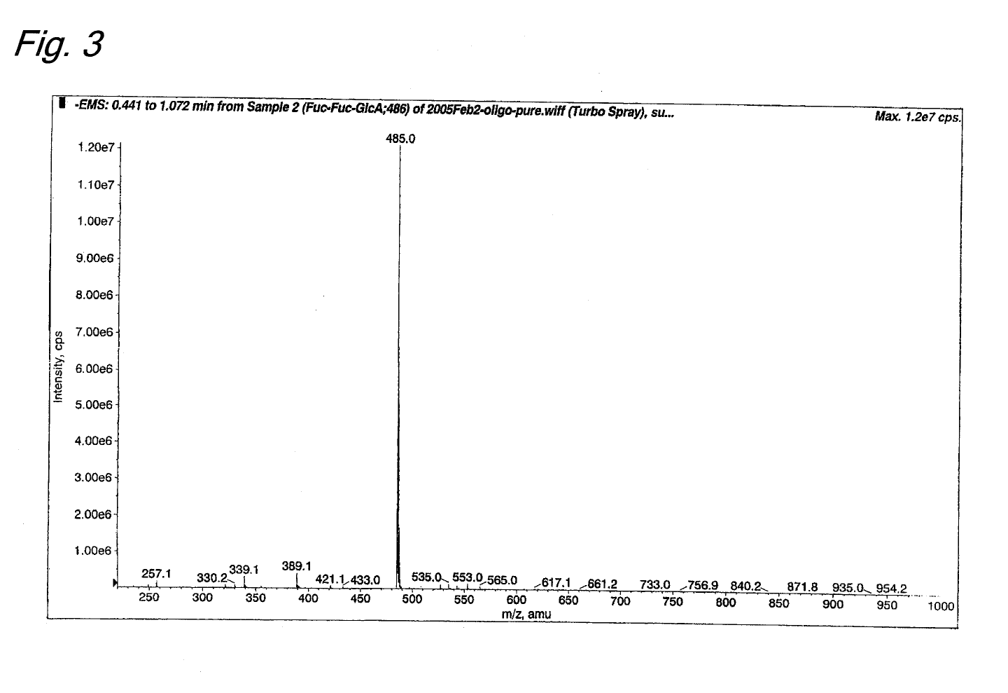

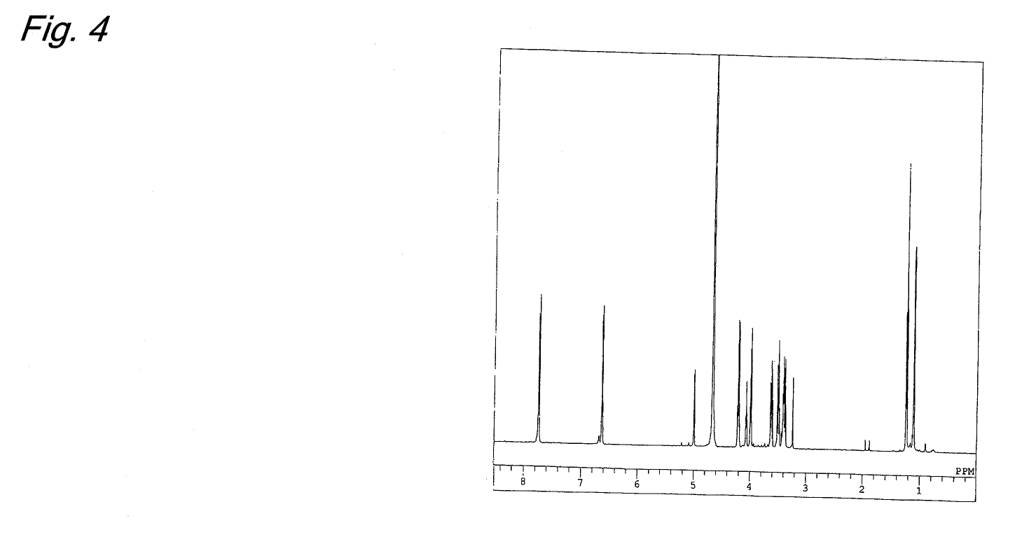

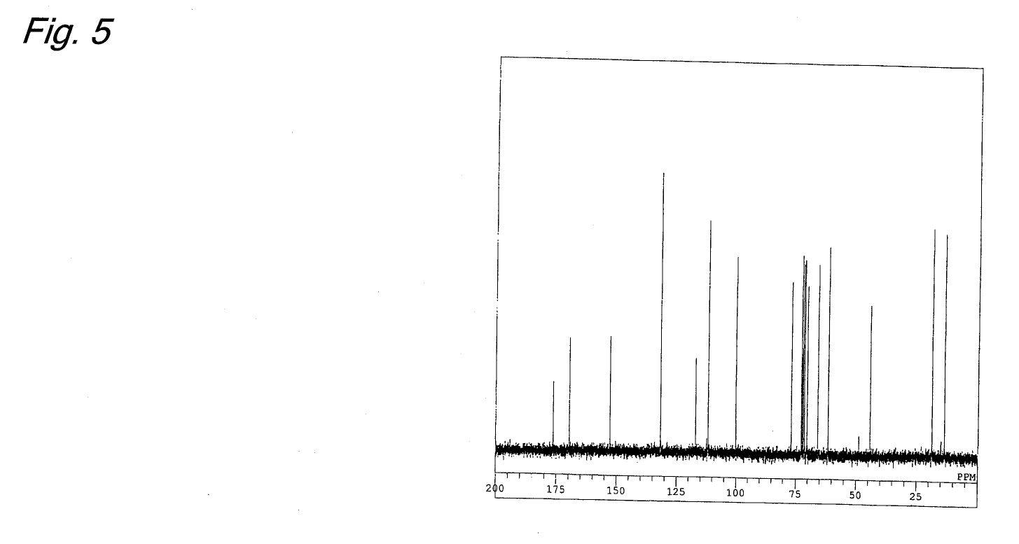

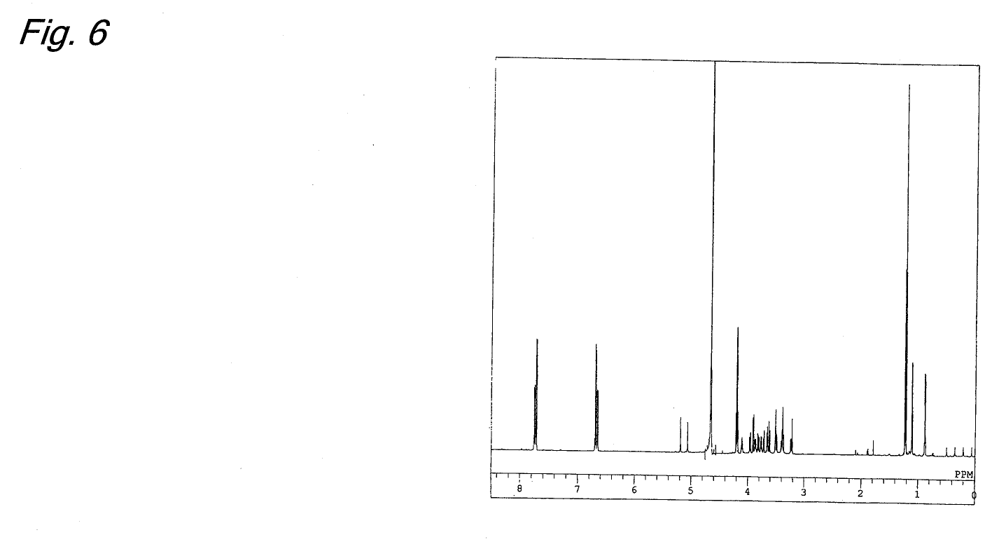

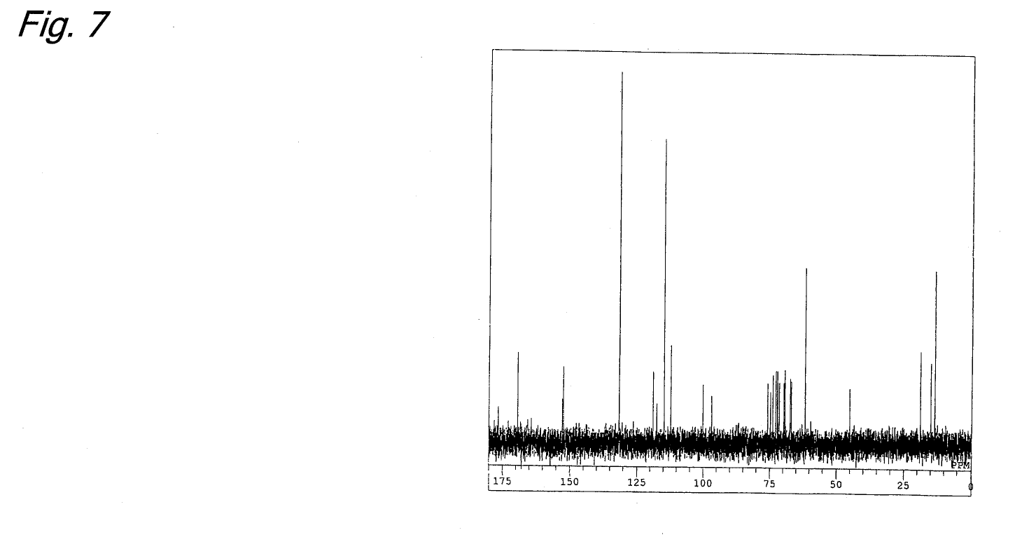

The fraction of neutral and acidic sugars obtained in b) (100 mg) was subjected to gel filtration (Biogel P-4 (Bio-Rad), elution solvent: aqueous solution of 0.2 M potassium borate (K₂B₄O₇)) to separate a disaccharide having a molecular weight of 340 and a trisaccharide having a molecular weight of 486 (compounds I, II) from the fractions. These molecular weights were determined by FAB-MS (FIGS. 2, 3; compound (I) [M-H]⁻: 339.2, compound (II) [M-H]⁻: 485.0). To 5 mg of these compounds, 1 ml of water, 1.6 g of ABEE (ethyl 4-aminobenzoate), 350 mg of NaBH₃CN (sodium cyanoborohydride), 3.5 ml of methanol, and 410 μl of acetic acid were added. The mixture was stirred at 65° C. for 4 hours. The reaction solution was partitioned between chloroform and water to obtain about 7 to 9 mg of the aforementioned disaccharide and trisaccharide that are fluorescently labeled. Charts of ¹H-NMR and ¹³C-NMR of these labeled oligosaccharides were shown in FIGS. 4 to 7, and the analysis results of them were shown in Tables 1 and 2. These results showed that the obtained disaccharide having a molecular weight of 340 was α-D-GlcA-(1→2)-L-Fuc represented by formula (I) and the trisaccharide having a molecular weight of 486 was α-D-GlcA-(1→2)-α-L-Fuc-(1→3)-L-Fuc represented by formula (II).

TABLE 1
¹H-NMR and ¹³C-NMR analysis results
of fluorescently labeled compound (I)

| | ¹H-NMR (D₂O) | ¹³C-NMR (D₂O) |
|---|---|---|
| Position | δ | δ |
| ABEE-1 | — | 117.0 |
| -2, 6 | 7.74(2H, d, J = 7.6 Hz) | 131.7 |
| -3, 5 | 6.63(2H, d, J = 7.6 Hz) | 111.8 |
| -4 | — | 152.8 |
| -C═O | — | 169.6 |
| -CH₂ | 4.21(2H, q, J = 14.3, 7.0, 1.5 Hz) | 61.6 |
| -CH₃ | 1.24(3H, td, J = 7.1, 1.6 Hz) | 13.6 |
| Fuc-1(CH₂) | 3.41(2H, m) | 44.3 |
| -2 | 4.07(1H, t, J = 6.4 Hz) | 77.1 |
| -3 | 3.56(1H, m) | 72.7 |
| -4 | 3.51(1H, m) | 72.8 |
| -5 | 3.99(1H, d, J = 8.6 Hz) | 66.0 |
| -CH₃ | 1.12(3H, d, J = 6.5 Hz) | 18.8 |
| GlcA-1 | 5.00(1H, d, J = 3.4 Hz) | 100.2 |
| -2 | 3.48(1H, m) | 71.6 |
| -3 | 3.63(1H, m) | 70.5 |
| -4 | 3.38(1H, m) | 72.0 |
| -5 | 4.00(1H, t, J = 6.4 Hz) | 72.4 |
| -COOH | — | 176.3 |

TABLE 2
¹H-NMR and ¹³C-NMR analysis results of
fluorescently labeled compound (II)

| | ¹H-NMR (D₂O) | ¹³C-NMR (D₂O) |
|---|---|---|
| Position | δ | δ |
| ABEE-1 | — | 117.6 |
| -2, 6 | 7.746 (2H, d, J = 8.9 Hz) | 131.6 |
| -3, 5 | 6.655 (2H, d, J = 8.6 Hz) | 112.4 |
| -4 | — | 152.9 |
| -C═O | — | 169.4 |
| -CH₂ | 4.191 (2H, q, J = 7.1 Hz) | 61.7 |
| -CH₃ | 1.220 (3H, t, J = 7.0 Hz) | 13.6 |
| Fuc-1(CH₂) | 3.396 (1H, q, J = 6.2 Hz) | 45.2 |
| | 3.223 (1H, q, J = 6.9 Hz) | |
| -2 | 4.115-4.089 (1H, m) | 69.8 |
| -3 | 3.713 (1H, q, J = 2.4 Hz) | 75.9 |
| -4 | 3.654 (1H, t, J = 4.6 Hz) | 73.9 |
| -5 | 3.866 (1H, dt, J = 12.3, 5.2 Hz) | 67.0 |
| -CH₃ | 1.103 (3H, d, J = 6.5 Hz) | 18.9 |
| Fuc-1 | 5.061 (1H, d, J = 4.1 Hz) | 97.0 |
| -2 | 3.812 (1H, dd, J = 10.3, 3.8 Hz) | 74.9 |
| -3 | 3.960 (1H, dd, J = 10.7, 3.4 Hz) | 69.4 |
| -4 | 3.506 (1H, t, J = 4.1 Hz) | 72.2 |
| -5 | 3.760 (1H, q, J = 6.5 Hz) | 67.4 |
| -CH₃ | 0.876 (3H, d, J = 6.5 Hz) | 15.1 |
| GlcA-1 | 5.187 (1H, d, J = 3.8 Hz) | 100.3 |
| -2 | 3.495 (1H, dd, J = 10.0, 4.1 Hz) | 71.5 |
| -3 | 3.624 (1H, t, J = 9.5 Hz) | 72.9 |
| -4 | 3.380 (1H, t, J = 9.6 Hz) | 72.0 |
| -5 | 3.903 (1H, d, J = 10.0 Hz) | 72.8 |
| -COOH | — | 176.6 |

### d) Production of Compounds (III) to (XI)

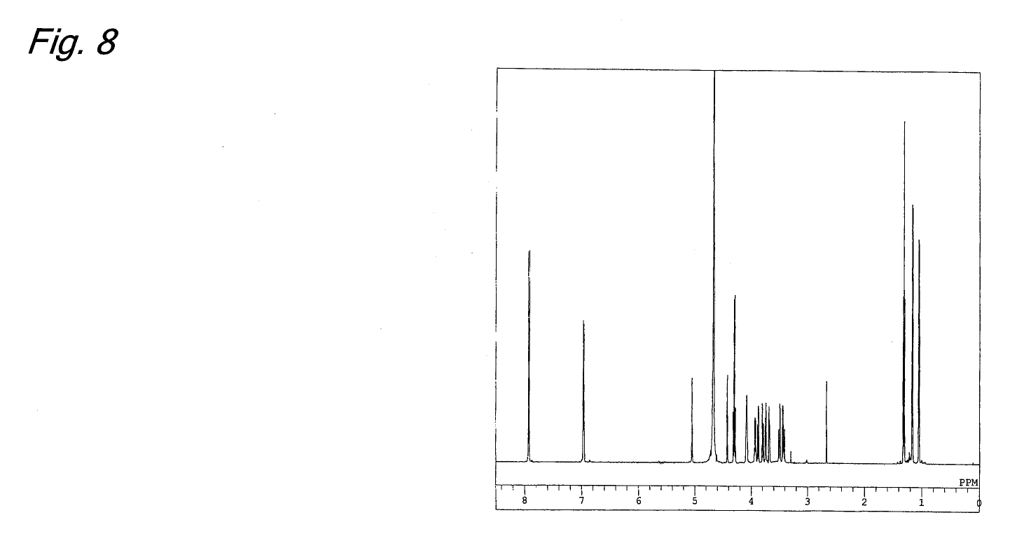

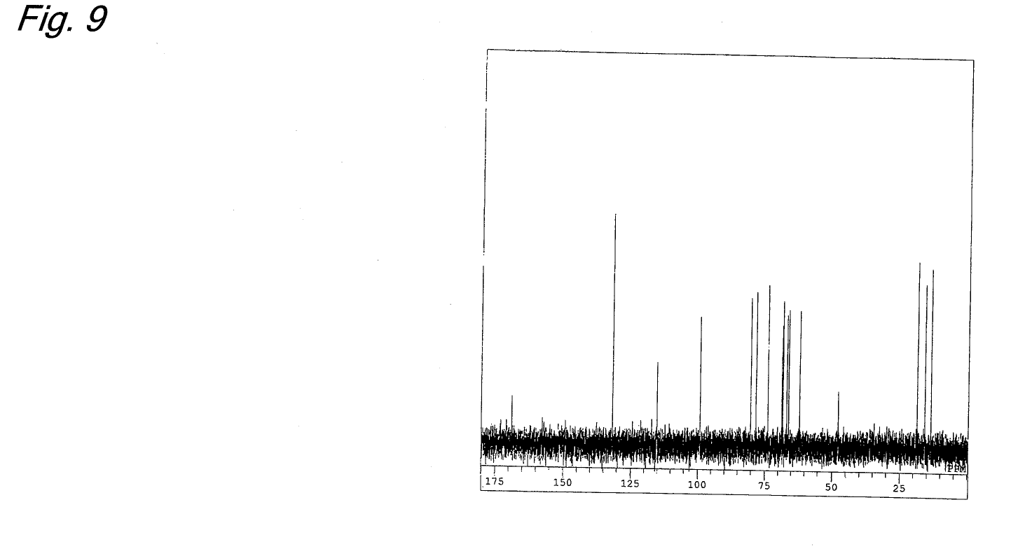

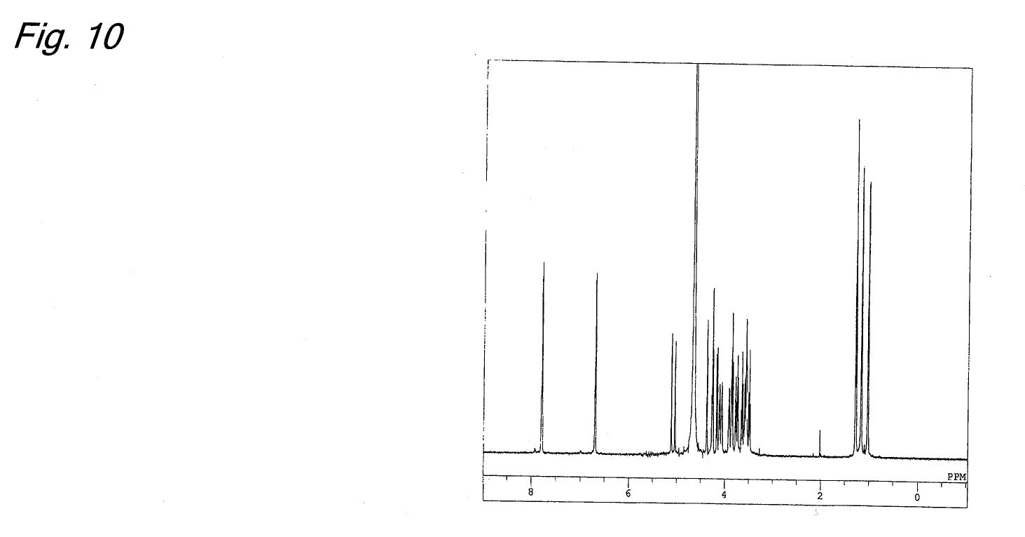

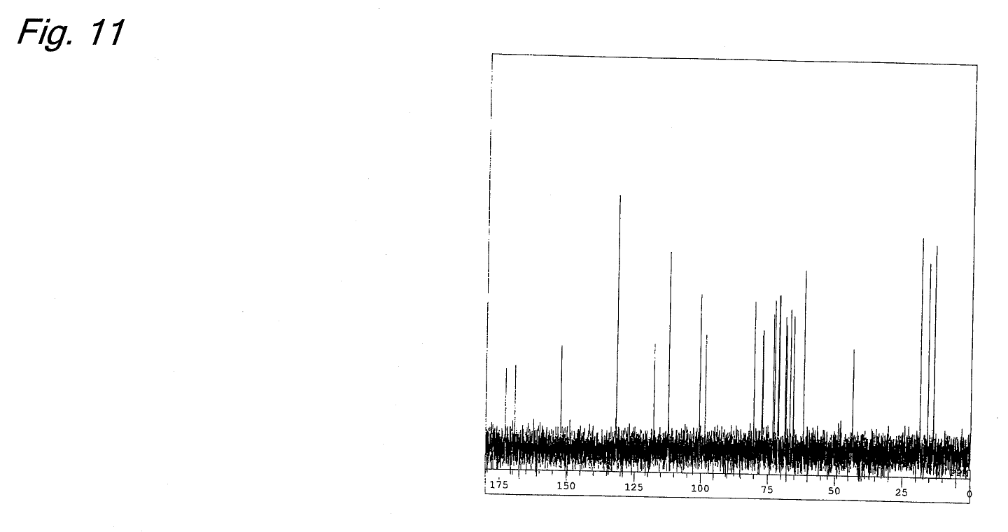

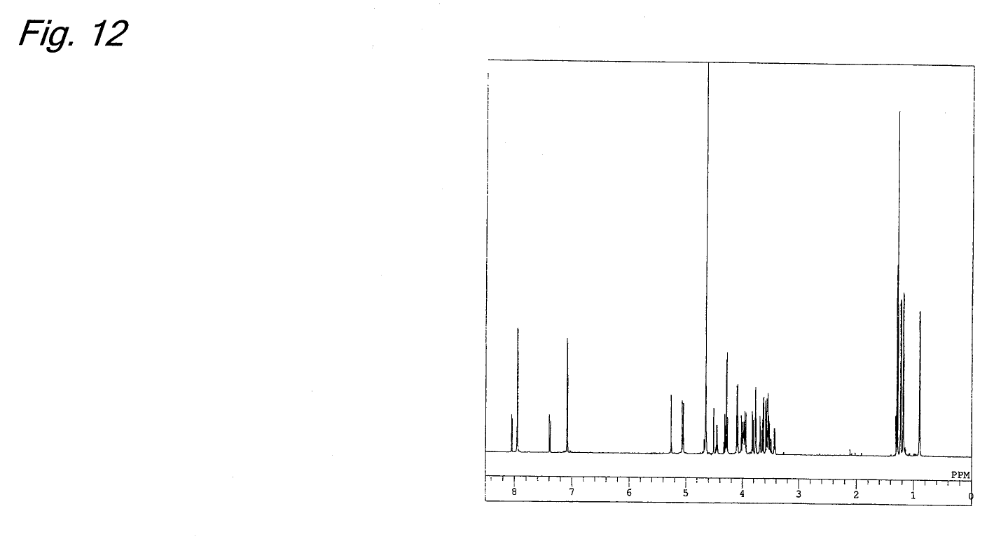

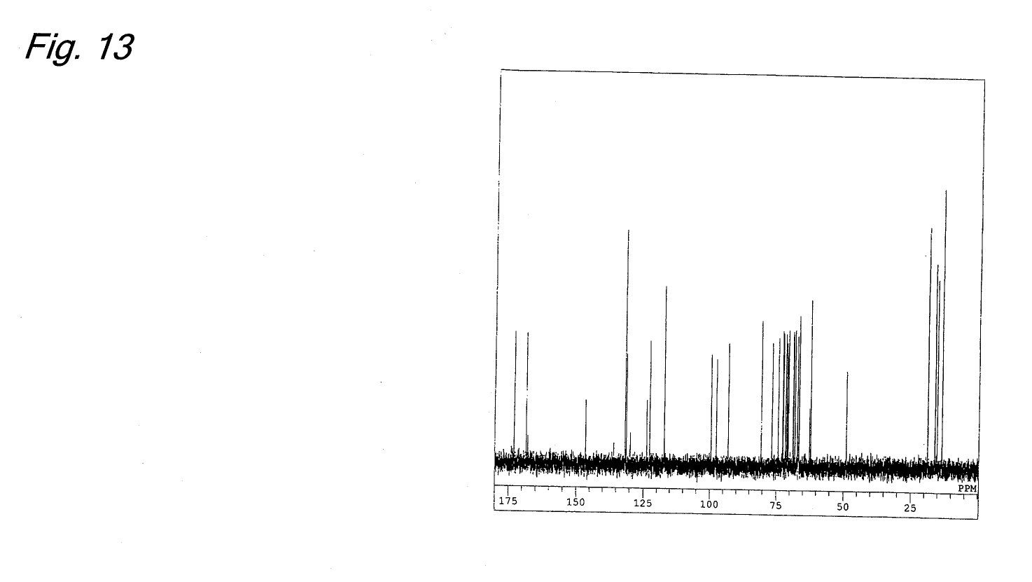

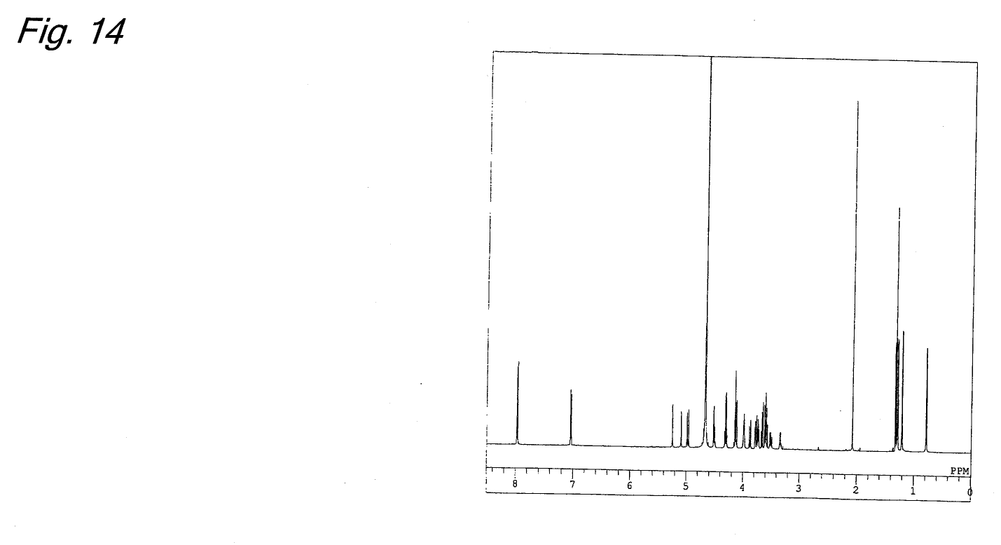

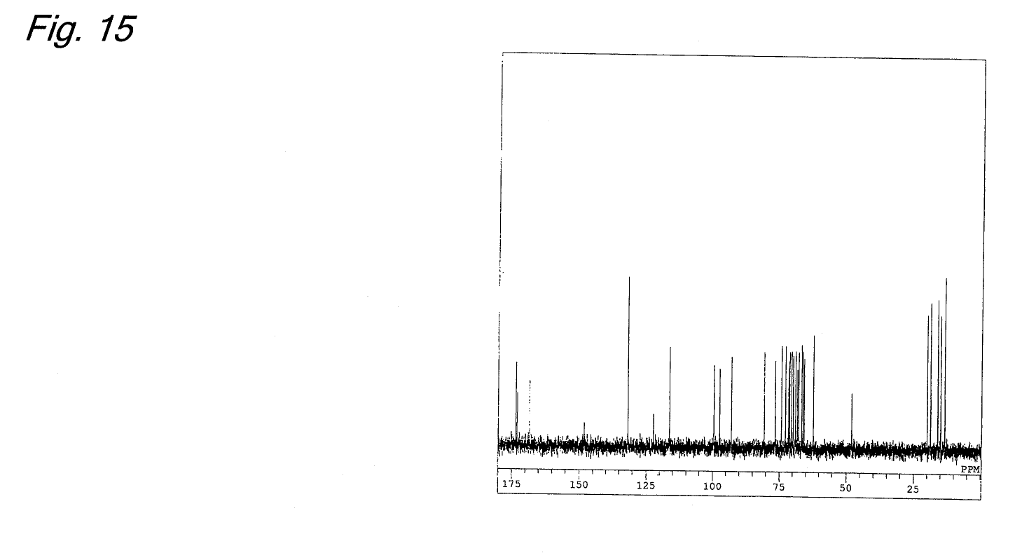

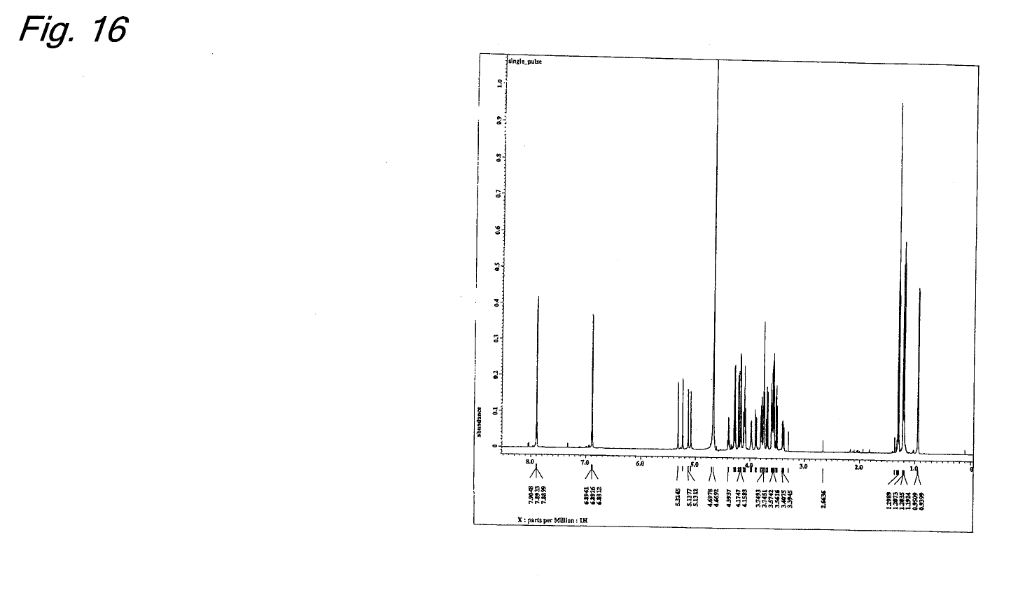

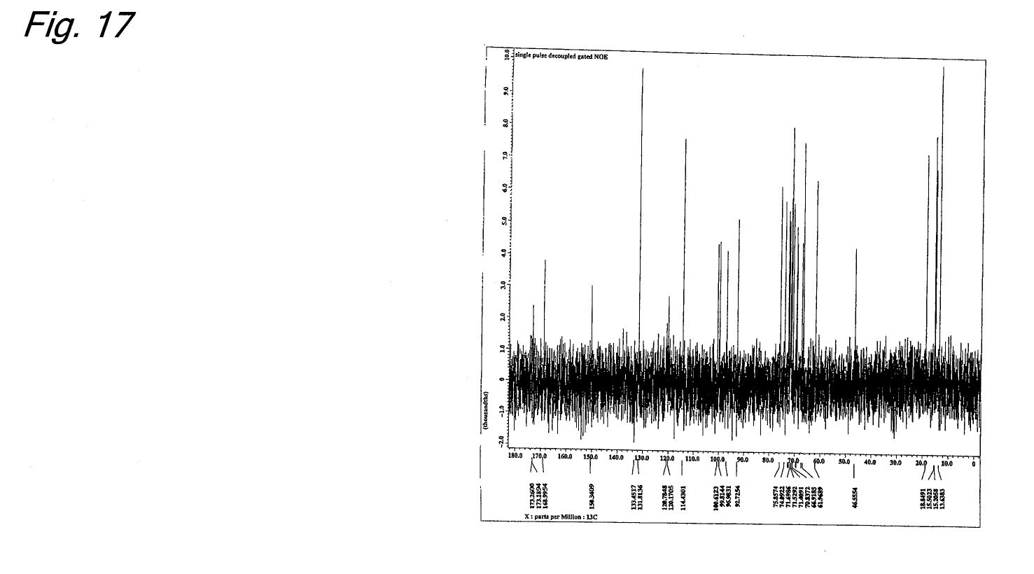

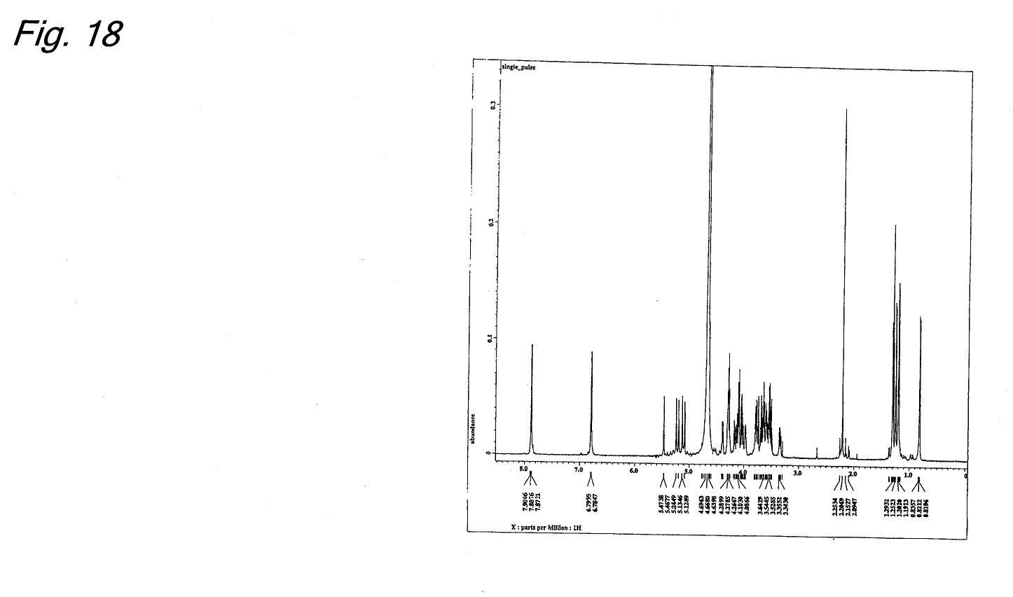

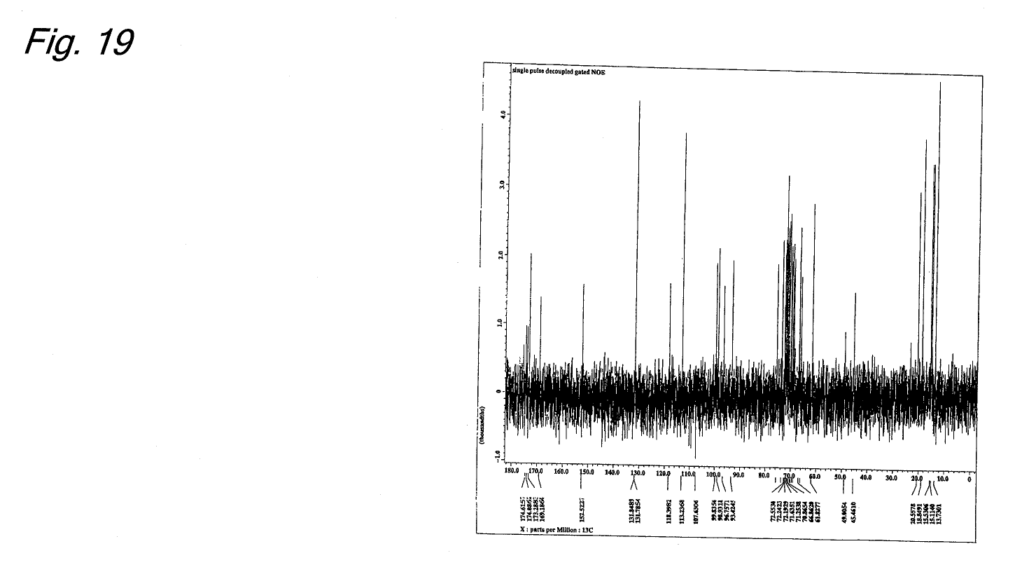

Next, the sulfated sugar fraction was subjected to gel filtration (Biogel P-6 (Bio-Rad)) for desalination. To 100 mg of the obtained sulfated sugar fraction, 1 ml of water, 1.6 g of ABEE (ethyl 4-aminobenzoate), 350 mg of NaBH₃CN (sodium cyanoborohydride), 3.5 ml of methanol, and 410 μl of acetic acid were added. The mixture was stirred at 65° C. for 4 hours. The obtained product was dried in vacuo, and partitioned between water and chloroform. The water layer was applied to a reverse phase column (carrier: Lichroprep RP-8 (25-40 μm) (Merck), 10 mmφ×220 mm; solvent condition: 5% CH₃CN/0.1% TFA (100 ml), 8% CH₃CN/0.1% TFA (100 ml), 15% CH₃CN/0.1% TFA (100 ml), 20% CH₃CN/0.1% TFA (100 ml)) to obtain a mixture of fluorescently labeled oligosaccharides. The obtained fluorescently labeled compounds were applied to HPLC (column: cosmosil 5C18-AR-II, 10.0 mmφ×250 mm; solvent condition: 12.5% CH₃CN/0.1% TFA (5 minutes), 12.5-27.5% CH₃CN/0.1% TFA (50 minutes); flow rate: 3 ml/min.) and eluted with acetonitrile:0.1% TFA aqueous solution with a concentration gradient of 5% to 30%. From the mixture, 6 labeled fucoidan oligosaccharides with sulfate groups having a molecular weight of 539, 715, 861, 903, 957, or 999, were separated (the molecular weights were determined by ESI-MS). NMR spectra of the obtained labeled oligosaccharides were determined and the results were analyzed. Charts of ¹H-NMR and ¹³C-NMR of the labeled oligosaccharides were shown in FIGS. 8 to 19, and their analysis results were shown in Tables 3 to 6. These results revealed that the compounds having a molecular weight of 539, 715, 861, 903, 957, and 999 were the labeled forms of compounds (III), (V), (VI), (VII), (VIII), and (IX), respectively.

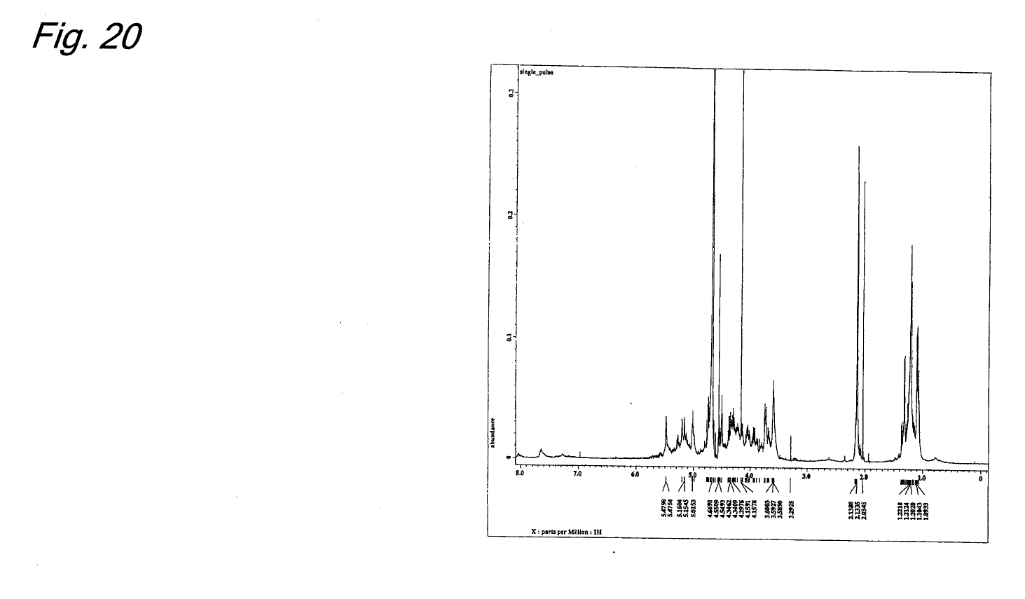

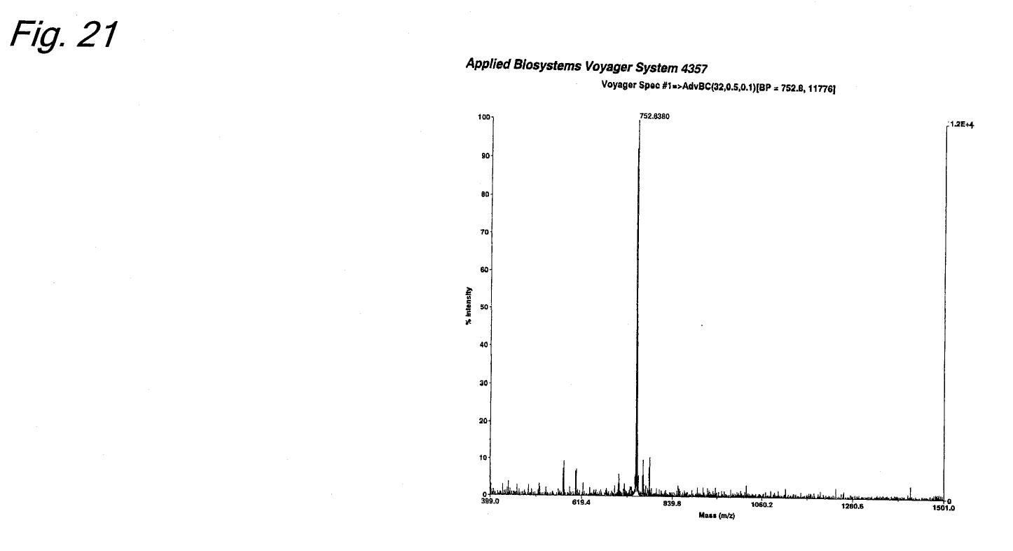

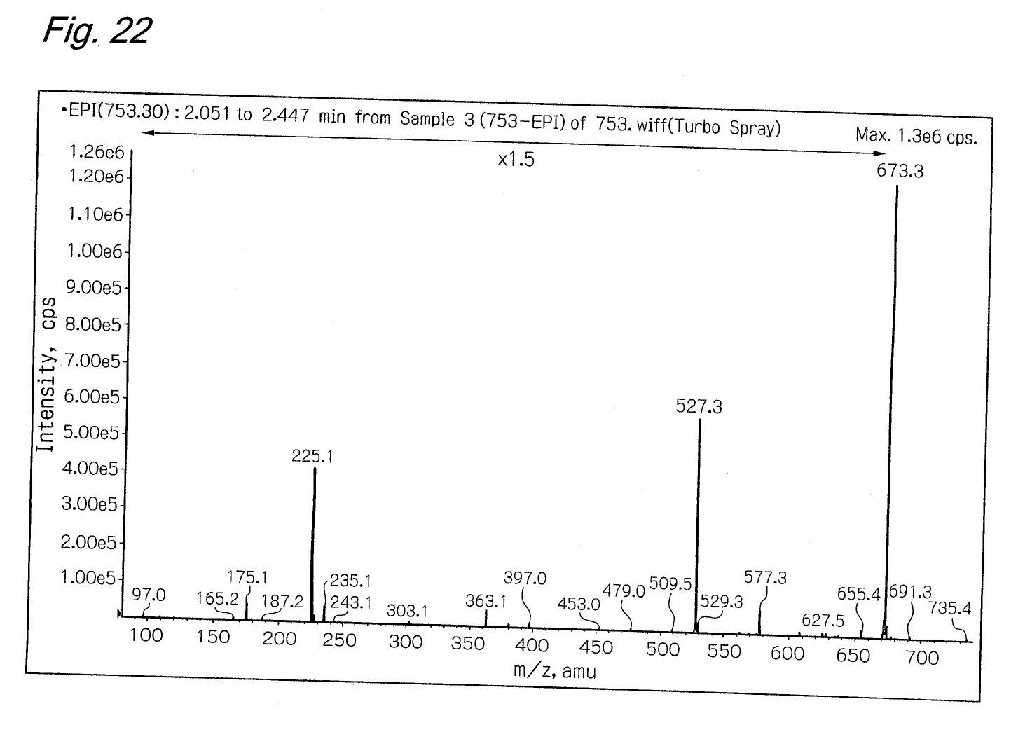

For confirmation, ABEE attached to each of the oligosaccharides was removed to reproduce pure oligosaccharides. That is, to 10 mg (100 μl) of each of these separated labeled oligosaccharides, 10 μl each of hydrogen peroxide and acetic acid were added. The mixture was allowed to stand for one day and night, and then dried. Among the thus obtained reproduced oligosaccharides, a saccharide obtained from a compound having a molecular weight of 903 was analyzed by ¹H-NMR (FIG. 20), TOF-MS (apparatus: Voyager DE-STR (Applied Biosystems), Ion mode: negative, Mode of operation: reflector, Accelerating voltage: 20 kV, Matrix: 2,5-dihydroxybenzoic acid) (FIG. 21), MS/MS (FIG. 22). The results surely showed the structure of formula (VII).

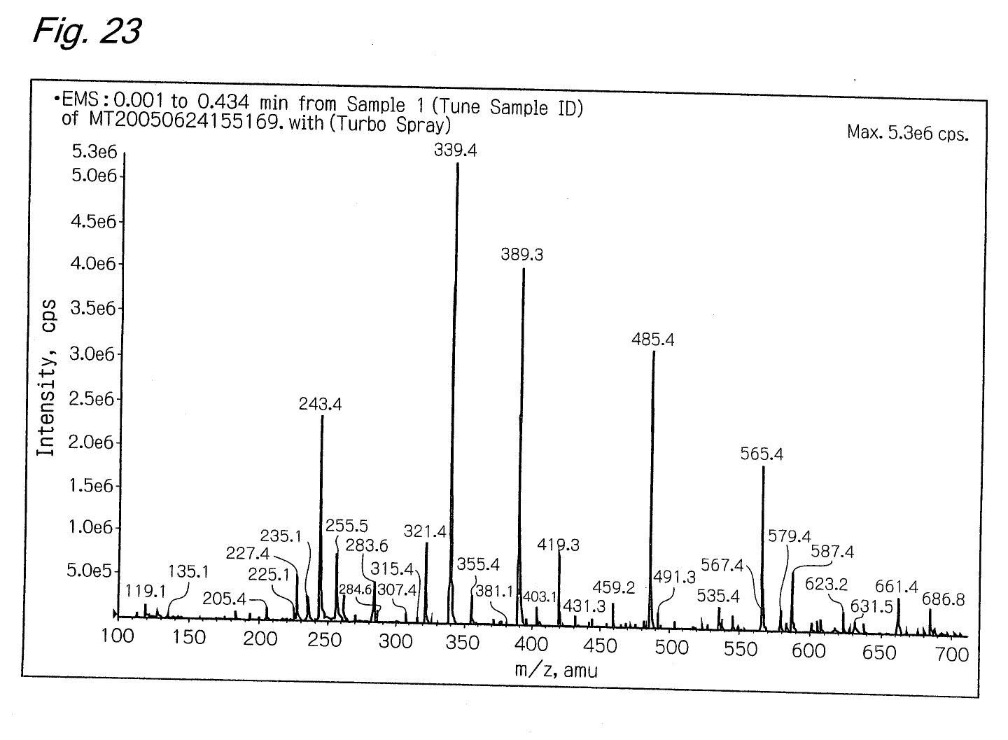

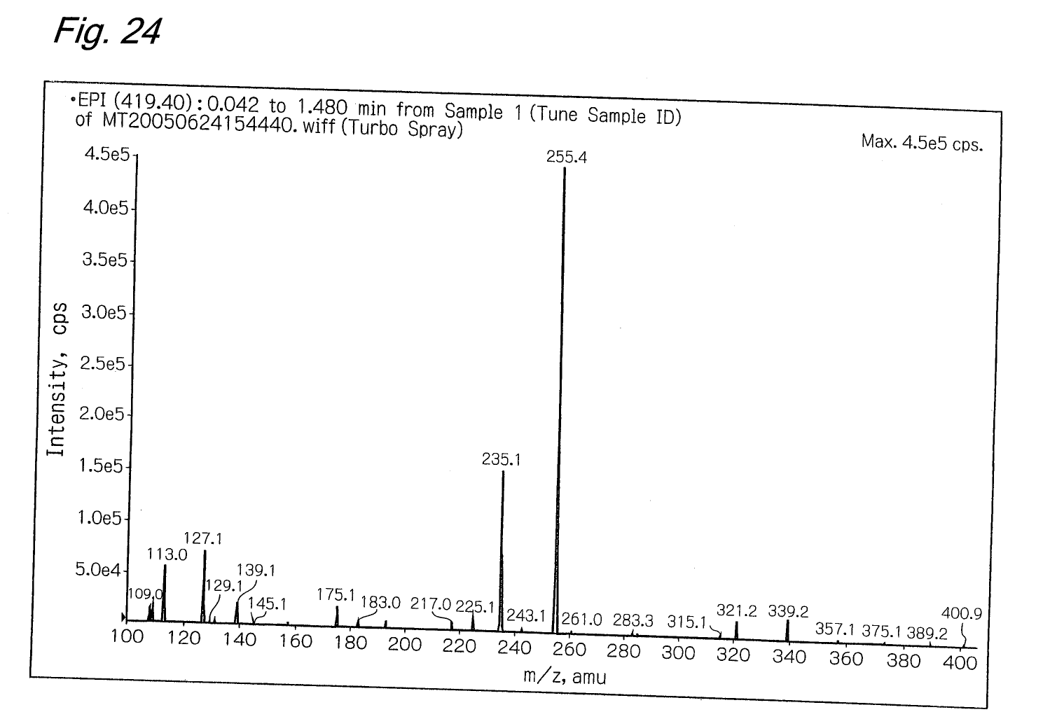

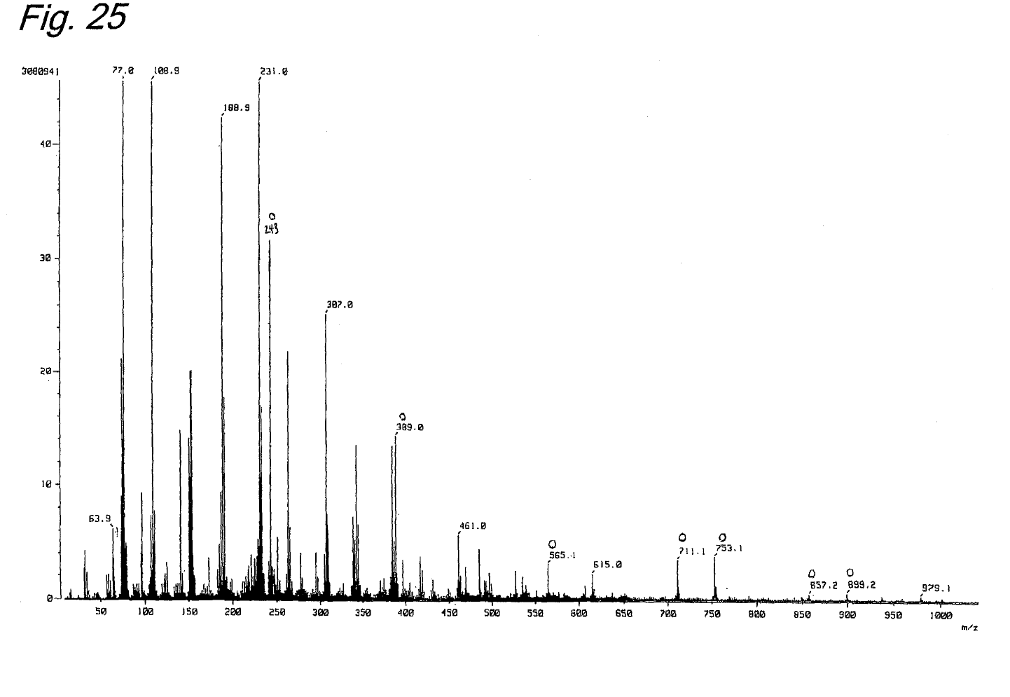

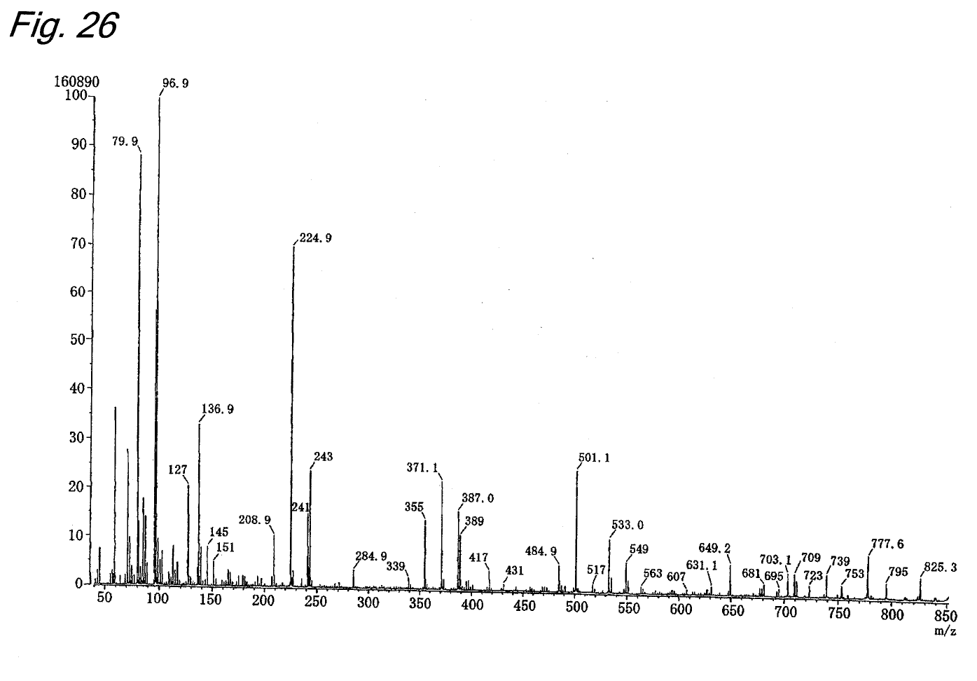

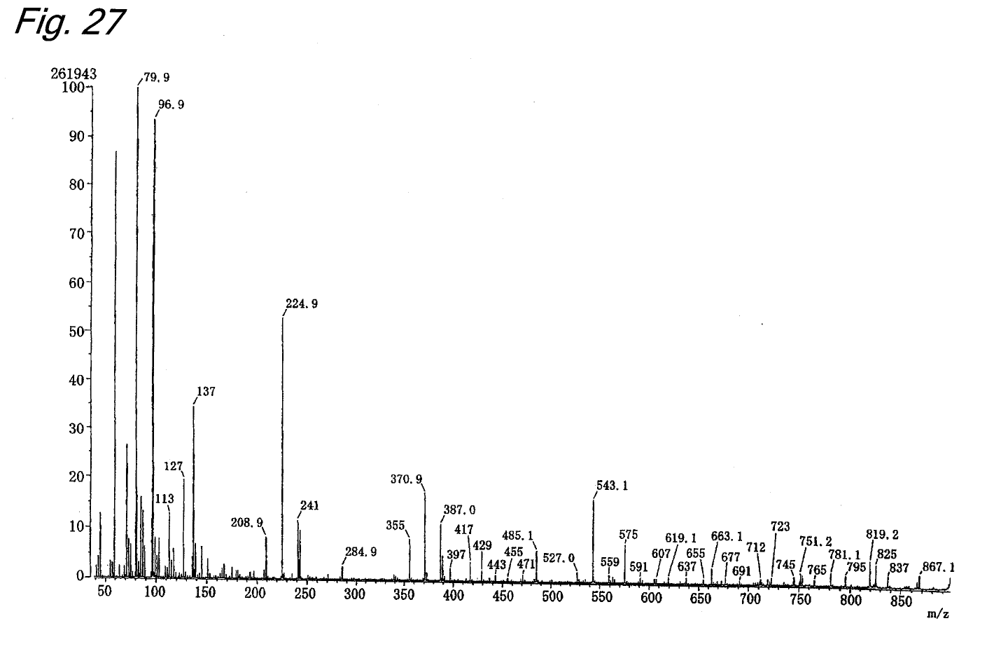

With regard to compounds having a molecular weight of 420, 858, or 900 ((IV), (X), (XI), respectively), which were not able to be separated by the aforementioned method, the existences were confirmed by analyzing the reaction mixture with FAB-MS/MS (apparatus: HX110A/HX110A (JEOL), Ion mode: MS, MS/MS (negative), Xe atom beam: 5 kV, Ion source accelerating potential: 10 kV, Collision energy: 2 keV, Matrix: Glycerol), and ESI-MS-MS. The analysis results of these unlabeled oligosaccharides are shown in FIGS. 23 to 27. FIG. 23 shows an FAB-MS chart of (IV), and FIG. 24 shows an MS/MS chart of (IV). FIG. 25 shows FAB-MS charts of (X) and (XI), and FIGS. 26 and 27 show their respective MS/MS charts.

These results revealed that a disaccharide having a molecular weight of 390 is α-L-Fuc-4-O—SO₃H⁻-(1→3)-L-Fuc represented by chemical formula (III), a disaccharide having a molecular weight of 420 is α-D-GlcA-(1→2)-L-Fuc represented by chemical formula (IV), a trisaccharide having a molecular weight of 566 is α-L-Fuc-4-O—SO₃H⁻-(1→3)-[α-D-GlcA-(1→2)]-L-Fuc represented by chemical formula (V), a tetrasaccharide having a molecular weight of 712 is α-L-Fuc-4-O—SO₃H⁻-(1→3)-[α-D-GlcA-(1→2)]-α-L-Fuc-(1→3)-L-Fuc represented by chemical formula (VI), a tetrasaccharide having a molecular weight of 754 is α-L-Fuc-4-O—SO₃H⁻-(1→3)-[α-D-GlcA-(1→2)]-α-L-Fuc-4-O-acetyl-(1→3)-L-Fuc represented by chemical formula (VII), a pentasaccharide having a molecular weight of 808 is [α-D-GlcA-(1→2)-α-L-Fuc-(1→3)]-[α-D-GlcA-(1→2)]-α-L-Fuc-(1→3)-L-Fuc represented by chemical formula (VIII), a pentasaccharide having a molecular weight of 850 is [α-D-GlcA-(1→2)-α-L-Fuc-(1→3)]-[α-D-GlcA-(1→2)]-4-O-acetyl-α-L-Fuc-(1→3)-L-Fuc represented by chemical formula (IX), a pentasaccharide having a molecular weight of 858 is α-L-Fuc-4-O—SO₃H⁻-(1→3)-α-L-Fuc-(1→3)-[α-D-GlcA-(1→2)]-α-L-Fuc-(1→3)-L-Fuc represented by chemical formula (X), and a pentasaccharide having a molecular weight of 900 is α-L-Fuc-4-O—SO₃H⁻-(1->3)-α-L-Fuc-(1→3)-[α-D-GlcA-(1→2)]-α-L-Fuc-4-O-acetyl-(1→3)-L-Fuc represented by chemical formula (XI).

TABLE 3
¹H-NMR and ¹³C-NMR analysis results
of fluorescently labeled compound (III)

| | ¹H-NMR (D₂O) | ¹³C-NMR (D₂O) |
|---|---|---|
| Position | δ | δ |
| ABEE-1 | | 117.3 |
| -2, 6 | 7.956 (2H, d, J = 8.9 Hz) | 131.8 |
| -3, 5 | 6.997 (2H, d, J = 8.7 Hz) | 115.9 |
| -4 | | 149.2 |
| -C═O | | 168.9 |
| -CH₂ | 4.331 (2H, q, J = 7.2 Hz) | 62.1 |
| -CH₃ | 1.343 (3H, t, J = 7.1 Hz) | 13.6 |
| Fuc-1(CH₂) | 3.530 (1H, dd, J = 13.4, 4.9 Hz) | 47.8 |
| | 3.456 (1H, dd, J = 13.5, 8.2 Hz) | |
| -2 | 4.113 (1H, dq, J = 12.0, 3.1 Hz) | 68.7 |
| -3 | 3.774 (1H, dd, J = 6.8, 1.5 Hz) | 78.4 |
| -4 | 3.709 (1H, dd, J = 6.2, 2.7 Hz) | 73.8 |
| -5 | 4.113 (1H, dq, J = 12.0, 3.1 Hz) | 66.1 |
| -CH₃ | 1.194 (3H, d, J = 6.4 Hz) | 18.7 |
| SFuc-1 | 5.081 (1H, d, J = 3.9 Hz) | 99.2 |
| -2 | 3.823 (1H, dd, J = 10.8, 4.1 Hz) | 68.3 |
| -3 | 3.909 (1H, dd, J = 10.5, 3.4 Hz) | 68.6 |
| -4-SO₃— | 4.455 (1H, d, J = 2.7 Hz) | 80.3 |
| -5 | 3.959 (1H, q, J = 6.3 Hz) | 66.8 |
| -CH₃ | 1.081 (3H, d, J = 6.6 Hz) | 15.9 |

TABLE 4
¹H-NMR and ¹³C-NMR analysis results
of fluorescently labeled compound (V)

| | ¹H-NMR (D₂O) | ¹³C-NMR (D₂O) |
|---|---|---|
| Position | δ | δ |
| ABEE-1 | — | 117.8 |
| -2, 6 | 7.794 (2H, d, J = 8.5 Hz) | 131.8 |
| -3, 5 | 6.699 (2H, d, J = 8.2 Hz) | 112.4 |
| -4 | — | 152.3 |
| -C═O | — | 169.4 |
| -CH₂ | 4.257 (2H, q, J = 7.1 Hz) | 61.8 |
| -CH₃ | 1.285 (3H, t, J = 7.1 Hz) | 13.7 |
| Fuc-1(CH₂) | 3.595 (1H, dd, J = 14.8, 4.7 Hz) | 43.6 |
| | 3.557 (1H, t, J = 3.5 Hz) | |
| -2 | 4.076-4.055 (1H, m) | 77.0 |
| -3 | 3.860 (1H, d, J = 8.7 Hz) | 77.3 |
| -4 | 3.729 (1H, d, J = 8.5 Hz) | 73.2 |
| -5 | 4.109 (1H, q, J = 6.5 Hz) | 65.6 |
| -CH₃ | 1.173 (3H, d, J = 6.4 Hz) | 18.6 |
| GlcA-1 | 5.111 (1H, d, J = 3.4 Hz) | 98.5 |
| -2 | 3.556-3.526 (1H, m) | 70.9 |
| -3 | 3.644 (1H, t, J = 9.2 Hz) | 72.6 |
| -4 | 3.489 (1H, t, J = 9.5 Hz) | 71.3 |
| -5 | 4.165 (1H, d, J = 10.3 Hz) | 71.2 |
| -COOH | — | 172.8 |
| SFuc-1 | 5.036 (1H, d, J = 3.7 Hz) | 100.6 |
| -2 | 3.768 (1H, dd, J = 10.8, 3.7 Hz) | 68.2 |
| -3 | 3.843 (1H, t, J = 5.5 Hz) | 68.6 |
| -4-SO₃— | 4.371 (1H, m) | 80.3 |
| -5 | 3.911 (1H, q, J = 6.4 Hz) | 66.9 |
| -CH₃ | 1.038 (3H, d, J = 6.4 Hz) | 15.8 |

TABLE 5
¹H-NMR and ¹³C-NMR analysis results of
fluorescently labeled compound (VI)

| | ¹H-NMR (D₂O) | ¹³C-NMR (D₂O) |
|---|---|---|
| Position | δ | δ |
| ABEE-1 | — | 123.8 |
| -2, 6 | 7.952 (2H, dd, J = 6.9, 2.1 Hz) | 131.4 |
| -3, 5 | 7.087 (2H, dd, J = 6.9, 1.8 Hz) | 117.3 |
| -4 | — | 146.6 |
| -C═O | — | 168.5 |
| -CH₂ | 4.286 (2H, q, J = 7.0 Hz) | 62.7 |
| -CH₃ | 1.292 (3H, t, J = 7.1 Hz) | 13.6 |
| Fuc-1(CH₂) | 3.526 (1H, dd, J = 13.4, 8.6 Hz) | 48.9 |
| | 3.572-3.556 (1H, m) | |
| -2 | 4.009-3.985 (1H, m) | 68.6 |
| -3 | 3.783 (1H, d, J = 2.7 Hz) | 76.8 |
| -4 | 3.708 (1H, dd, J = 6.0, 3.7 Hz) | 74.4 |
| -5 | 4.009-3.985 (1H, m) | 66.6 |
| -CH₃ | 1.185 (3H, d, J = 6.4 Hz) | 18.9 |
| Fuc-1 | 5.049 (1H, d, J = 3.7 Hz) | 97.7 |
| -2 | 4.117 (1H, d, J = 9.8 Hz) | 70.9 |
| -3 | 4.033 (1H, dd, J = 10.6, 2.9 Hz) | 72.5 |
| -4 | 3.792 (1H, d, J = 2.7 Hz) | 66.8 |
| -5 | 3.430 (1H, q, J = 6.7 Hz) | 67.2 |
| -CH₃ | 0.900 (3H, d, J = 6.6 Hz) | 15.3 |
| SFuc-1 | 5.069 (1H, d, J = 3.9 Hz) | 93.2 |
| -2 | 3.821 (1H, dd, J = 10.4, 3.8 Hz) | 68.1 |
| -3 | 3.970 (1H, dd, J = 10.4, 3.1 Hz) | 68.9 |
| -4-SO₃— | 4.512 (1H, d, J = 3.0 Hz) | 80.8 |
| -5 | 4.455 (1H, q, J = 6.6 Hz) | 66.6 |
| -CH₃ | 1.232 (3H, d, J = 6.4 Hz) | 16.2 |
| GlcA-1 | 5.266 (1H, d, J = 3.7 Hz) | 99.6 |
| -2 | 3.602 (1H, dd, J = 9.7, 4.0 Hz) | 70.6 |
| -3 | 3.643 (1H, t, J = 9.4 Hz) | 72.7 |
| -4 | 3.587-3.556 (1H, m). | 71.2 |
| -5 | 4.108 (1H, t, J = 5.4 Hz) | 71.6 |
| -COOH | — | 172.92 |

TABLE 6
¹H-NMR and ¹³C-NMR analysis results
of fluorescently labeled compound (VII)

| | ¹H-NMR (D₂O) | ¹³C-NMR (D₂O) |
|---|---|---|
| Position | δ | δ |
| ABEE-1 | — | 122.4 |
| -2, 6 | 7.953 (2H, dd, J = 7.0, 1.7 Hz) | 131.9 |
| -3, 5 | 7.021 (2H, d, J = 8.9 Hz) | 116.4 |
| -4 | — | 148.2 |
| -C═O | — | 168.5 |
| -CH₂ | 4.290 (2H, ddd, J = 14.3, 7.1, 1.3 Hz) | 62.2 |
| -CH₃ | 1.299 (3H, t, J = 7.1 Hz) | 13.6 |
| Fuc-1(CH₂) | 3.604-3.573 (1H, m) | 48.0 |
| | 3.488 (1H, dd, J = 13.5, 8.0 Hz) | |
| -2 | 3.977-3.953 (1H, m) | 68.9 |
| -3 | 3.766 (1H, q, J = 2.9 Hz) | 76.6 |
| -4 | 3.713 (1H, td, J = 5.8, 3.8 Hz) | 74.3 |
| -5 | 3.977-3.953 (1H, m) | 66.7 |
| -CH₃ | 1.191 (3H, d, J = 6.4 Hz) | 18.8 |
| FucAc-1 | 5.078 (1H, d, J = 2.7 Hz) | 97.5 |
| -2 | 4.120 (1H, d, J = 1.8 Hz) | 71.3 |
| -3 | 4.120 (1H, d, J = 1.8 Hz) | 69.9 |
| -4 | 4.971 (1H, s) | 68.3 |
| -5 | 3.319 (1H, d, J = 6.6 Hz) | 65.8 |
| -COCH₃ | 2.058 (3H, s) | 173.5/20.1 |
| -CH₃ | 0.764 (3H, d, J = 6.4 Hz) | 15.1 |
| GlcA-1 | 5.231 (1H, d, J = 3.7 Hz) | 99.7 |
| -2 | 3.604-3.573 (1H, m) | 70.5 |
| -3 | 3.644 (1H, t, J = 9.4 Hz) | 72.8 |
| -4 | 3.556 (1H, d, J = 8.9 Hz) | 71.2 |
| -5 | 4.102 (1H, t, J = 4.9 Hz) | 71.5 |
| -COOH | — | 173.0 |
| SFuc-1 | 4.947 (1H, d, J = 4.1 Hz) | 93.1 |
| -2 | 3.740 (1H, d, J = 3.9 Hz) | 67.8 |
| -3 | 3.858 (1H, dd, J = 10.5, 3.2 Hz) | 68.9 |
| -4-SO₃— | 4.501 (1H, d, J = 3.9 Hz) | 80.8 |
| -5 | 4.485 (1H, t, J = 7.4 Hz) | 66.2 |
| -CH₃ | 1.267 (3H, d, J = 6.6 Hz) | 16.2 |

### Example 2

### Preparation of Fucoidan Oligosaccharide-2

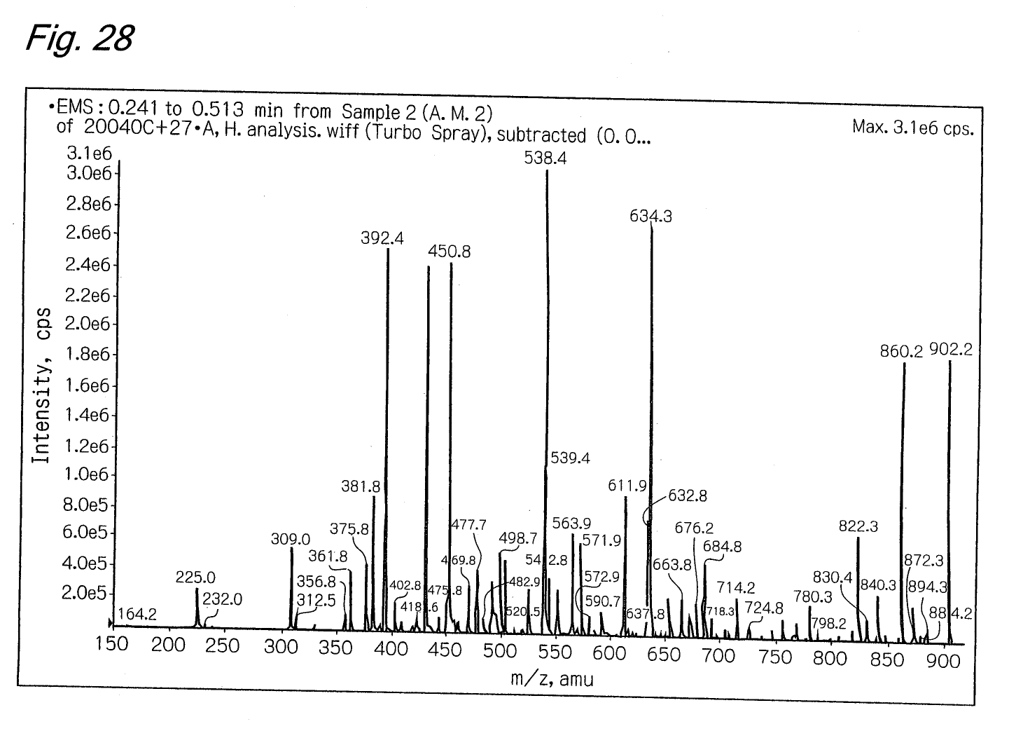

To 100 g of Okinawa Nemacystus decipiens, 1000 ml of 2 N HCl was added, and the mixture was subjected to acid hydrolysis at 50° C. to 100° C. for 1 hour. The obtained extract was cooled, and then filtered by suction, electrodialyzed (desalinated), and lyophilized to obtain 2 g of fucoidan fractions. The fucoidan fractions were fluorescently labeled by ABEE, and then analyzed by ESI-MS (4000Q TRAP LC/MS/MS system (Applied Biosystems); analysis conditions, Polarity: Negative ion mode; Declustering Potential: −50 v; Collision energy: −10 eV; Temperature: 550° C.). As a result, a chart shown by FIG. 28 was obtained, and the existences of fucoidan oligosaccharides represented by formulae (I) to (XI) were confirmed.

### Example 3

### Preparation of Sulfated Fucose-Free Fucoidan Oligosaccharide

1. Fucoidan (10 g, Okinawa Hakko Kagaku, Japan) was added to 200 ml of 1 N HCl and hydrolyzed at 70° C. to 105° C. for 15 to 30 minutes while stirring in a medium bottle.
2. After cooling, the hydrolysate was neutralized with NaOH and filtered. When filtration took a long time, centrifugation was performed for solid-liquid separation, and the liquid phase was then filtered.
3. To the filtrate, powdered activated carbon was added and stirred at ordinary temperature for 15 minutes, followed by filtration through a 0.45 μm Millipore filter to remove the activated carbon.
4. Using a Micro Acilyzer G3 (Asahi Kasei Corporation, Japan), desalination was performed with an AC110 membrane to reach a constant conductivity.
5. To 100 ml of a strongly acidic cation exchange resin Diaion SK1B (H-type, Mitsubishi Chemical Corporation, Japan), the whole volume (about 300 ml) was loaded and then washed with water (100 ml) to collect the entire eluate (water-eluted fraction). Cations such as metal ions were removed by being adsorbed on the resin.
6. The entire water-eluted fraction was loaded onto 120 ml of a weakly basic anion exchange resin Diaion WA30 (OH-type, Mitsubishi Chemical Corporation, Japan) and eluted with water (600 ml) to obtain neutral sugars (e.g., fucose, xylose) and then with 10% formic acid (500 ml) to obtain sulfated fucose-free acidic oligosaccharides, followed by elution with 0.5 N HCl (300 ml), 1 N HCl (300 ml) and 3 N HCl (300 ml) to obtain sulfated fucose-containing acidic oligosaccharides.
7. The sulfated fucose-free acidic oligosaccharide fraction eluted with 10% formic acid (500 ml) was concentrated under reduced pressure to remove formic acid. This solution was analyzed by HPLC as described in Example 7-i to confirm peaks of GF (I), GF2 (II), G2F2 (XII) and G2F3 (VIII). This solution was lyophilized to give a powder.
8. The 1 N HCl-eluted fraction (sulfated fucose-containing acidic oligosaccharide fraction) was concentrated under reduced pressure. This fraction was analyzed by HPLC as described in Example 7-i to detect sulfated fucose, GSF (V) as a trisaccharide, as well as GSF2 (VI) and GSFaF (VII) as tetrasaccharides.
9. A lyophilized product of the 10% formic acid-eluted fraction was dissolved in water, mixed with powdered activated carbon, stirred for 20 minutes and then filtered through a 0.45 μm Millipore filter to remove colored components.
10. The filtrate was ultrafiltered with an ultrafiltration apparatus equipped with an Amicon filter YM10 (a membrane with a molecular weight cutoff of 10,000), and the resulting filtrate was concentrated to 3 ml under reduced pressure.
11. The concentrated solution was loaded in 4 portions (0.5 ml, 0.625 ml, 0.625 ml and 0.625 ml) onto an NH2 column (Asahipak NH2-P-90 (20×300 mm) and eluted for 70 minutes with CH3CN:50 mM HCl=4:1 at 6 ml/min while heating a column oven at 50° C. and then further eluted for 50 minutes with CH3CN:50 mM HCl=3:1 at 6 ml/min. The absorbance at 210 nm was measured to collect peaks corresponding to GF (I), GF2 (II), G2F2 (XII) and G2F3 (VIII).
12. Each of the fractions was concentrated under reduced pressure and then neutralized with NaOH, followed by desalination to reach a constant conductivity using a Micro Acilyzer S1 (Asahi Kasei Corporation, Japan) with AC112 as a membrane. Each of the desalinated fractions was concentrated under reduced pressure and then lyophilized.

As a result, sodium salts were obtained for GF (I), GF2 (II), G2F2 (XII) and G2F3 (VIII).

### Example 4

### Preparation of Sulfated Fucose-Containing Fucoidan oligosaccharide

1. In the same manner as used in Example 3, fucoidan (Okinawa Hakko Kagaku, Japan) was hydrolyzed, neutralized with NaOH, filtered, treated with activated carbon, and then desalinated with a Micro Acilyzer.
2. To 100 ml of a strongly acidic cation exchange resin Diaion SK1B (H-type, Mitsubishi Chemical Corporation, Japan), the whole volume (about 250 ml) was loaded and then washed with water (60 ml) to collect the entire eluate (water-eluted fraction). Cations such as metal ions were removed by being adsorbed on the resin.
3. The entire water-eluted fraction was loaded onto 200 ml of a weakly basic anion exchange resin Diaion WA30 (OH-type, Mitsubishi Chemical Corporation, Japan) and eluted with water (1000 ml) to obtain neutral sugars (e.g., fucose, xylose) and then with 10% formic acid (1000 ml) to obtain sulfated fucose-free acidic oligosaccharides, followed by elution with 0.2 N HCl (600 ml), 0.4 N HCl (750 ml) and 1 N HCl (1000 ml) to obtain sulfated fucose-containing acidic oligosaccharides.
4. The latter half (520 ml) of the 0.4 N HCl-eluted fraction (sulfated fucose-containing acidic oligosaccharide fraction) was concentrated to 50 ml under reduced pressure to remove hydrochloric acid.
5. The concentrated solution was neutralized with 1 N NaOH and then desalinated to reach a constant conductivity using a Micro Acilyzer G3 (Asahi Kasei Corporation, Japan) with AC110 as a membrane.
6. The desalinated solution was filtered with an ultrafiltration apparatus equipped with an Amicon filter YM10 (a membrane with a molecular weight cutoff of 10,000) and washed with water. The filtrate and washing solution were concentrated under reduced pressure and lyophilized to give a dry powder. This fraction was analyzed by HPLC as described in Example 7-i to detect sulfated fucose, GSF (V) as a trisaccharide, as well as GSFF (VI) and GSFaF (VII) as tetrasaccharides.
7. The dry powder was loaded in 3 portions onto an NH2 column (Asahipak NH2-P-90, 20 mmφ×300 mm) and eluted for 150 minutes with CH3CN:133 mM HCl=7:3 at 6 ml/min while heating a column oven at 50° C.
8. The fractions eluted around 80 minutes (containing VII) and around 135 minutes (containing V and VI) were concentrated under reduced pressure and then neutralized with NaOH, followed by desalination to reach a constant conductivity using a Micro Acilyzer S1 (Asahi Kasei Corporation, Japan) with AC110 as a membrane. Each of the desalinated fractions was concentrated under reduced pressure and then lyophilized.

As a result, sodium salts were obtained for the VII-containing fraction and the fraction containing V and VI.

### Example 5

### Preparation of Sulfated Fucose

1. In the same manner as used in Example 3, fucoidan (Okinawa Hakko Kagaku, Japan) was hydrolyzed, neutralized with NaOH, filtered, treated with activated carbon, and then desalinated with a Micro Acilyzer. The desalinated solution was loaded onto a cation exchange resin Diaion SK1B (H-type) to collect the eluate (water-eluted fraction). Cations such as metal ions were removed by being adsorbed on the resin.
2. The entire water-eluted fraction was loaded onto 200 ml of a weakly basic anion exchange resin WA30 (OH-type) and eluted with water (1000 ml) to obtain neutral sugars (e.g., fucose, xylose) and then with 10% formic acid (1000 ml) to obtain sulfated fucose-free acidic oligosaccharides, followed by elution with 0.2 N HCl (600 ml), 0.4 N HCl (750 ml) and 1 N HCl (1000 ml) to obtain sulfated fucose-containing acidic oligosaccharides.
3. The first half (250 ml) of the 1 N HCl-eluted fraction (sulfated fucose fraction) was concentrated to 50 ml under reduced pressure.
4. The concentrated solution was neutralized with 1 N NaOH and then desalinated to reach a constant conductivity using a Micro Acilyzer G3 (Asahi Kasei Corporation, Japan) with AC110 as a membrane.
5. The desalinated solution was filtered with an ultrafiltration apparatus equipped with an Amicon filter YM10 (a membrane with a molecular weight cutoff of 10,000), and the filtrate was concentrated under reduced pressure. This fraction was analyzed by HPLC as described in Example 7-i, indicating that it was sulfated fucose.
6. The concentrated solution (0.5 ml) was loaded in two portions onto an NH2 column (Asahipak NH2-P-90, 20 mmφ×300 mm) and eluted for 150 minutes with CH3CN:133 mM HCl=7:3 at 6 ml/min while heating a column oven at 50° C. The R1 absorbance was measured to collect a peak corresponding to sulfated fucose eluted around 100 minutes.
7. This fraction was concentrated under reduced pressure and then neutralized with NaOH, followed by desalination to reach a constant conductivity using a Micro Acilyzer S1 (Asahi Kasei Corporation, Japan) with AC110 as a membrane. The desalinated fraction was concentrated under reduced pressure and then lyophilized.

As a result, a sodium salt was obtained for sulfated fucose.

### Example 6

### Preparation of Fucoidan Oligosaccharide

1. Fucoidan (60 g, Okinawa Hakko Kagaku, Japan) was mixed with 1200 ml of 1 N HCl and hydrolyzed at 70° C. to 105° C. for 15 minutes to 3 hours in a medium bottle.
2. After cooling, the hydrolysate was neutralized with NaOH and filtered. When filtration took a long time, centrifugation was performed for solid-liquid separation, and the liquid phase was then filtered.
3. To the filtrate, powdered activated carbon was added and stirred at ordinary temperature for 15 minutes, followed by filtration through a 0.45 μm Millipore filter to remove the activated carbon.
4. Using a Micro Acilyzer G3 (Asahi Kasei Corporation, Japan), desalination was performed with an AC110 membrane to reach a constant conductivity, thereby obtaining a clouded desalinated solution.
5. The clouded desalinated solution was filtered through a 0.45 μm Millipore filter to remove the cloudiness.
6. To 100 ml of a strongly acidic cation exchange resin Diaion SK1B (H-type, Mitsubishi Chemical Corporation, Japan), the whole volume was loaded and eluted, followed by washing with water (100 ml) to collect the entire eluate (water-eluted fraction). Cations such as metal ions were removed by being adsorbed on the resin.
7. The entire water-eluted fraction was concentrated to 250 ml under reduced pressure, 100 ml of which was then loaded onto 120 ml of a weakly basic anion exchange resin Diaion WA30 (OH-type, Mitsubishi Chemical Corporation, Japan) and eluted with water (475 ml) to obtain neutral sugars (e.g., fucose, xylose) and then with 10% formic acid (500 ml) to obtain sulfated fucose-free acidic oligosaccharides, followed by elution with 1 N HCl (500 ml) to obtain sulfated fucose-containing acidic oligosaccharides.
8. The sulfated fucose-free acidic oligosaccharide fraction eluted with 10% formic acid (500 ml) was concentrated under reduced pressure to remove formic acid. This solution was analyzed by HPLC as described in Example 7-i to confirm a peak of GF(I). This solution was lyophilized to give a white powder.

### Example 7

### Purity Measurement and Qualitative Analysis on Isolated Product

i. HPLC Method

The GF (I), GF2 (II), G2F2 (XII) and G2F3 (VIII) samples obtained in Example 3 were each prepared into a 5% aqueous solutions and analyzed under the following conditions: column: CAPCELLPAK-NH2 (4.6 mmφ×250 mm, Shiseido Co., Ltd., Japan); column temperature: 60° C.; mobile phase: acetonitrile:100 mM HCl=75:25 or 70:30 at 1 ml/min; and detection: RI and UV (210 nm).

As a result, for GF (I), a peak was detected at 9.048 minutes in RI detection, and its RI purity was about 98%, excluding its sodium salt detected at 4.3 minutes.

For GF2 (II), a peak was detected at 10.55 minutes in RI detection, and its RI purity was about 93%, excluding its sodium salt detected at 4.3 minutes.

For G2F2 (XII), a peak was detected at 18.29 minutes in RI detection, and its RI purity was about 77%, excluding its sodium salt detected at 4.3 minutes. The remaining 23% was G2F3 (VIII).

For G2F3 (VIII), a peak was detected at 21.569 minutes in RI detection, and its RI purity was about 78%, excluding its sodium salt detected at 4.3 minutes. The remaining 22% was G2F2 (XII).

ii. Qualitative Analysis by MS

GF (I), GF2 (II), G2F2 (XII) and G2F3 (VIII) obtained in Example 3 were each dissolved at about 10 ppm in 50% methanol/H₂O, and then measured with a Q-TOF (Micromass, UK) using a Z-spray, an ESI ion source and a nanocapillary in the negative mode. The capillary voltage was set at 1000 V and the cone energy was set at 30 V for measurement. As a result, GF, GF2, G2F2 and G2F3 showed ions indicative of the oligosaccharides at m/z 339[M-H]−, m/z 485[M-H]−, m/z 661[M-H]- and m/z 807[M-H]−, respectively.

iii. Structural Analysis by Labeling and NMR
Preparation of Labeled Sugar with ABEE (4-aminobenzoic acid ethyl ester)

### [Reaction]

1) 4.280 mg of the G2F2=XII fraction (obtained in Example 3) was dissolved in 85.6 μL water to give a 5 wt % aqueous solution. To this solution, 320 μL ABEE reagent solution* (ABEE reagent solution*: 165 mg (1.0 mmol) ethyl 4-aminobenzoate, 34 mg (0.58 mmol) dimethylamine borane, 350 μL methanol, 41 μL acetic acid) was added and stirred, followed by reaction at 65° C. for 1 hour.
2) To the reaction solution, chloroform (1.6 mL)-water (1.6 mL) was added to repeat liquid-liquid extraction three times.
3) The combined aqueous layers were solid-phase extracted on a SepPakC18 (eluent: 30% acetonitrile/water) and then lyophilized to give a powder (2.8 mg).

### [Preparative HPLC Conditions]

Column: YMC-Pack ODS-AM-323 S-5 μm (10 mmφ×250 mm)

Mobile phase: A: H₂O-0.1% HCOOH, B: CH3CN-0.1% HCOOH

Flow rate: 2.0 mL/min

Gradient: B conc. 8% isocratic (20 minutes), B 8%→30% (40 minutes)

Detection: A305 nm

Fractions eluted between 45 and 47 minutes (ABEE-labeled G2F2) were collected and lyophilized (0.6 mg).

### [Instrumental Analysis on ABEE-Labeled G2F2 (XII)]

The ABEE-labeled G2F2 obtained by preparative HPLC was analyzed by mass spectrometry with an LCMS-IT-TOF (Shimadzu Corporation, Japan) using an ESI ion source in the negative mode. As a result, the [M-H]− ion was detected at m/z 810.2624, and the molecular formula was determined to be C33H49O22N (with an error of 5.43 ppm from the calculated molecular weight of 810.2668).

Next, ABEE-labeled G2F2=XII was dissolved in CD3OD and analyzed by NMR with an AVANCE-750 spectrometer (BRUKER BIOSPIN, Germany). The items measured were ¹H-NMR, COSY, TOCSY, HSQC and HMBC.

As a result of structural analysis by MS and NMR, the oligosaccharide moiety was found to be [α-D-GlcA-(1→2)-α-L-Fuc-(1→3)]-[α-D-GlcA-(1→2)]-L-Fuc represented by formula (XII).

### Example 8

### Measurement of α-glucosidase Activity

1. 0.1 M sodium phosphate buffer (a mixture of 0.1 M NaH2PO4.2H₂O and 0.1 M Na2HPO4.12H₂O, adjusted to pH 7.0) was supplemented with 2 g/L bovine serum albumin (a product of Nacalai Tesque, Inc., Japan, F-V, pH 5.2, purity: 96%) and 0.2 g/L NaN3 (a product of Nacalai Tesque, Inc., Japan, reagent grade). As an enzyme solution, α-glucosidase (a product of Wako Pure Chemical Industries, Ltd., Japan, derived from yeast, 100 units/mg) was dissolved in the above buffer at 0.5 units/mg protein/ml (100 μg/20 ml). As a substrate solution, p-nitrophenyl-α-D-glucopyranoside (a product of Nacalai Tesque, Inc., Japan, reagent grade) was dissolved in the above buffer at 5 mM (7.525 mg/5 ml).
2. For use as samples, GF (I) purified in Example 3 was adjusted to 200 mg/ml H₂O and diluted two-fold to give 6 dilutions. Using a 96-well microplate, the enzyme solution (45 μL) was added to each of the sample solutions (10 μL) and pre-incubated at 37° C. for 5 minutes. After addition of the substrate solution (45 μL), each sample was measured for absorbance A405 nm (A405 nm at 0 min) and, after incubation at 37° C. for 5 minutes, was then measured for absorbance A405 nm (A405 nm at 5 min). As a control, H₂O was added in place of the samples and measured for absorbance. The difference in A405 nm from the control was calculated as % inhibition. Activity measurement was made in quadruplicate.
3. For use as samples, GF (I), GF2 (II), G2F2 (XII) and G2F3 (VIII) purified in Example 3, the GSFaF (VII)-containing fraction and the fraction containing GSF and GSFF (V & VI) prepared in Example 4, as well as sulfated fucose (S) purified in Example 5 were each adjusted to 50 mg/ml H₂O. As comparative samples, X2 (xylobiose) and glucuronic acid (Sigma-Aldrich) were neutralized with NaOH and adjusted to 50 mg/ml H₂O (calculated based on the amount of glucuronic acid). Using a 96-well microplate, the enzyme solution (45 μL) was added to each of the sample solutions (10 μL) and pre-incubated at 37° C. for 5 minutes. After addition of the substrate solution (45 μl), each of the samples was measured for absorbance A405 nm (A405 nm at 0 min) and, after incubation at 37° C. for 5 minutes, was then measured for absorbance A405 nm (A405 nm at 5 min). As a control, H₂O was added in place of the samples, and the difference in A405 nm from the control was calculated as % inhibition. Activity measurement was made in duplicate.
4. Calculation equation

Calculation was based on the following equations.

(A405 nm at 5 min in control)−(A405 nm at 0 min in control)=ΔA405 nm(cont.)

(A405 nm at 5 min in sample)−(A405 nm at 0 min in sample)=ΔA405 nm(sample)

{ΔA405 nm(cont.)−ΔA405 nm(sample)}/ΔA405 nm(cont.)×100=% inhibition

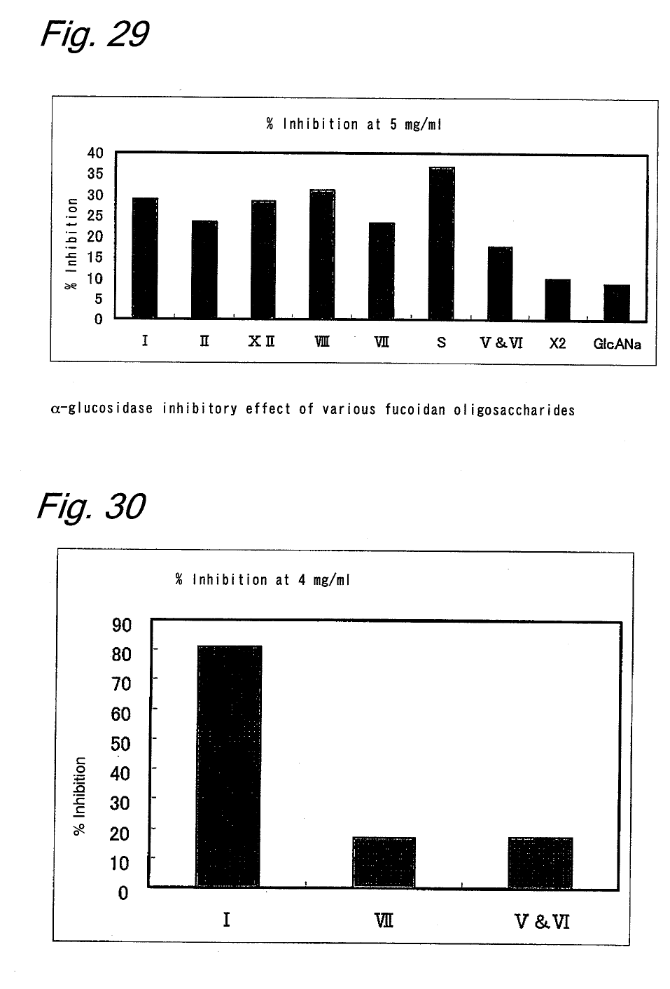

5. The results of 2 indicated that GF (I) inhibited α-glucosidase in a dose-dependent manner and had an IC50 of 20.4 mg/ml.
6. The results of 3 indicated that among fucoidan-derived oligosaccharides, those consisting of glucuronic acid and fucose (I, II, VIII, XII) showed 23% to 31% α-glucosidase inhibitory activity at 5 mg/ml, while sulfated fucose and those consisting of sulfated fucose, glucuronic acid and fucose showed 17% to 36% α-glucosidase inhibitory activity. These saccharides were found to have stronger activity than X2 (10.15%) which is known to cause α-glucosidase inhibition (Patent Document 8). Moreover, a saccharide whose constituent sugar, glucuronic acid, was neutralized, showed 8% inhibitory activity (FIG. 29).

### Example 9

### Measurement of Lipase Inhibitory Activity

1. In a 96-well flat-bottomed plate, GF (I) prepared in Example 3, as well as the GSFaF (VII)-containing fraction and the GSF fraction containing (V) and GSFF (VI) prepared in Example 4 were each adjusted to give a final concentration of 4 mg/ml (25 μl), followed by addition of 50 μl buffer (130 mM Tris-HCl buffer (pH 8.0, containing 150 mM NaCl and 1.36 mM CaCl₂)) and 25 μl 4-methylumbelliferone oleic acid ester (SIGMA, final concentration: 100 μM). The plate was allowed to stand for 30 minutes at room temperature. Then, lipase (porcine pancreatic lipase, SIGMA) was added in a volume of 50 μl (final concentration: 100 U/ml) to initiate the reaction.
2. After 30 minutes, 100 μl citrate buffer (pH 4.2) was added to stop the reaction. The fluorescence intensity of 4-methylumbelliferone generated by the reaction (excitation wavelength: 355 nm, fluorescence wavelength: 460 nm) was measured with a fluorescence plate reader (Fluoroskan Asent CF, Labsystems).
3. As a control, water was used in place of the samples. As a blank, water was used in place of the samples and buffer was used in place of lipase. The following equation was used to calculate lipase inhibitory activity.
4. Calculation equation

Lipase inhibitory activity (%)=100−(A−B)/(C−B)×100

A: fluorescence intensity in sample, B: fluorescence intensity in blank, C: fluorescence intensity in control

5. The results indicated that GF (I) showed 81% lipase inhibitory activity at 4 mg/ml, while the GSFaF (VII)-containing fraction ((VII) in FIG. 30) and the fraction containing GSF (V) and GSFF (VI) (GSF & GSFF in FIG. 30) showed around 17% lipase inhibitory activity at 4 mg/ml (FIG. 30).

### Example 10

### Evaluation of Quality of Taste

A sensory test at ordinary temperature was made by 4 panelists on a 5% aqueous solution of the sulfated fucose-free fucoidan oligosaccharide prepared in Example 3-7, a 5% aqueous solution of the sulfated fucose-containing fucoidan oligosaccharide prepared in Example 4-6, GF (I) prepared in Example 6, and fucoidan. The panelists were allowed to comment freely.

The results obtained are shown in Table 7. The sulfated fucose-free fucoidan oligosaccharide and the sulfated fucose-containing fucoidan oligosaccharide were found to have a faint sweetness. Moreover, fucoidan was a dark-brown solution with a residual seaweed odor, whereas GF was a colorless solution serving as a refreshing acidifier with a slight sweetness.

TABLE 7
Sensory test at ordinary temperature on 5% aqueous solutions
of sulfated fucose oligosaccharide, GF (I) and fucoidan

| | 5% Sulfated | 5% Sulfated | | |
|---|---|---|---|---|
| | fucose-free | fucose-con- | | |
| | fucoidan | taining fucoidan | | |
| Panel | oligosaccharide | oligosaccharide | 5% GF | 5% Fucoidan |
| A | | | Refreshing | Undaria-like |
| | | | sourness, | taste |
| | | | slight | |
| | | | sweetness | |
| B | Faint sweetness | Faintly sweet | Pleasant | Seaweed odor, |
| | and hop-like | | sourness | unpleasant |
| | refreshing | | | |
| | bitterness | | | |
| C | Bodied | Faintly sweet, | Sour | Laver flavor, |
| | sweetness | slightly salty | | bad aftertaste |
| D | Sweetness and | Faintly sweet | | |
| | glaze spreading | | | |
| | in the mouth | | | |

1. A compound, which comprises glucuronic acid (G), fucose (F), sulfated fucose (S) and acetylated fucose (Fa) in one molecule as shown below:
GF, GFF (GF2), SF, GS, GSF, GSFF (GSF2), GSFAaF, GGFFF (G2F3), GGFaFF (G2FaF2), GSFFF (GSF3), GSFaFFF (GSFaF3) or GGFF (G2F2).

2. A compound represented by the following structural formula (I), (II), (III), (IV), (V), (VI), (VII), (VIII), (IX), (X), (XI) or (XII):

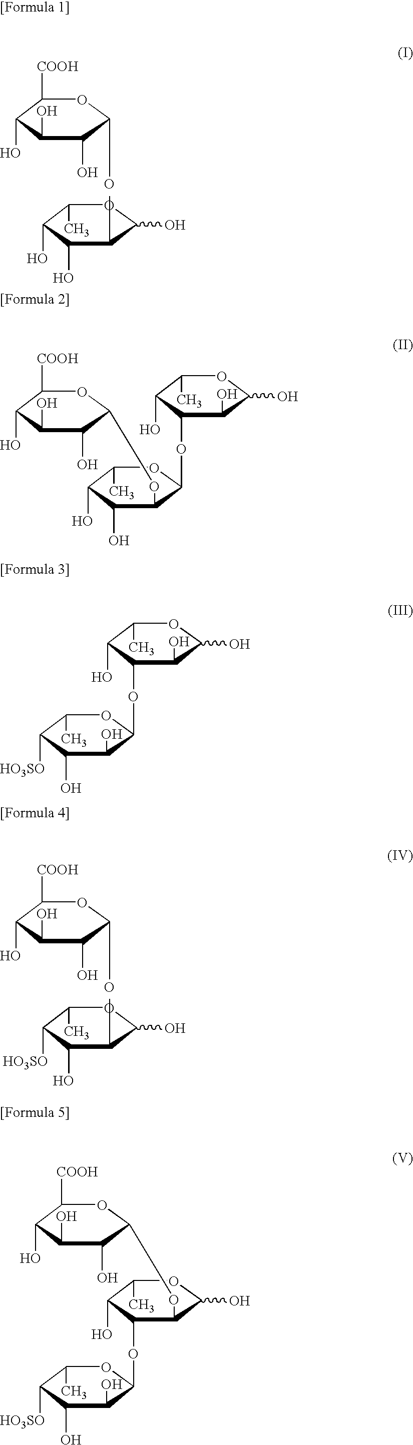

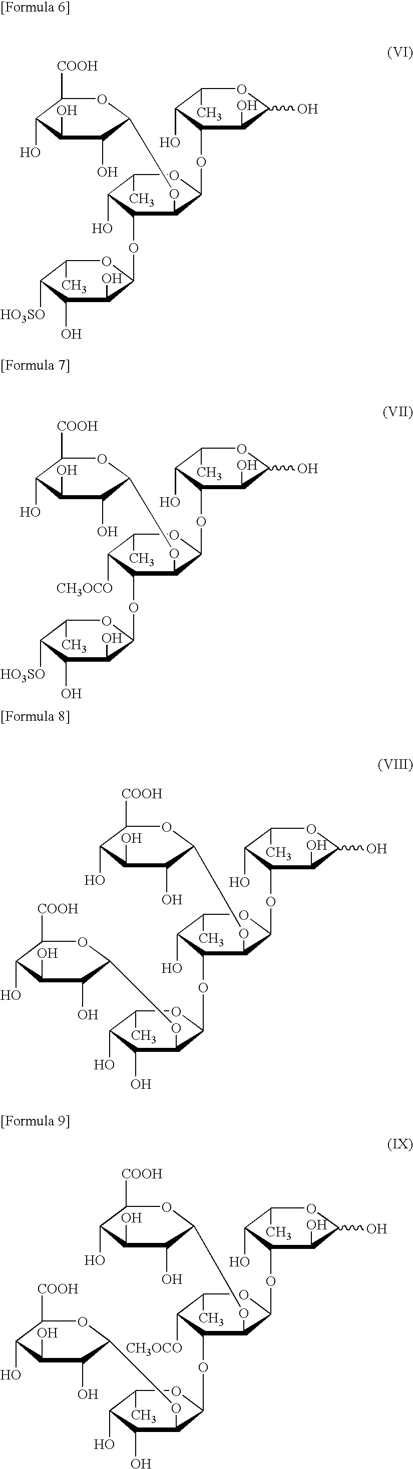

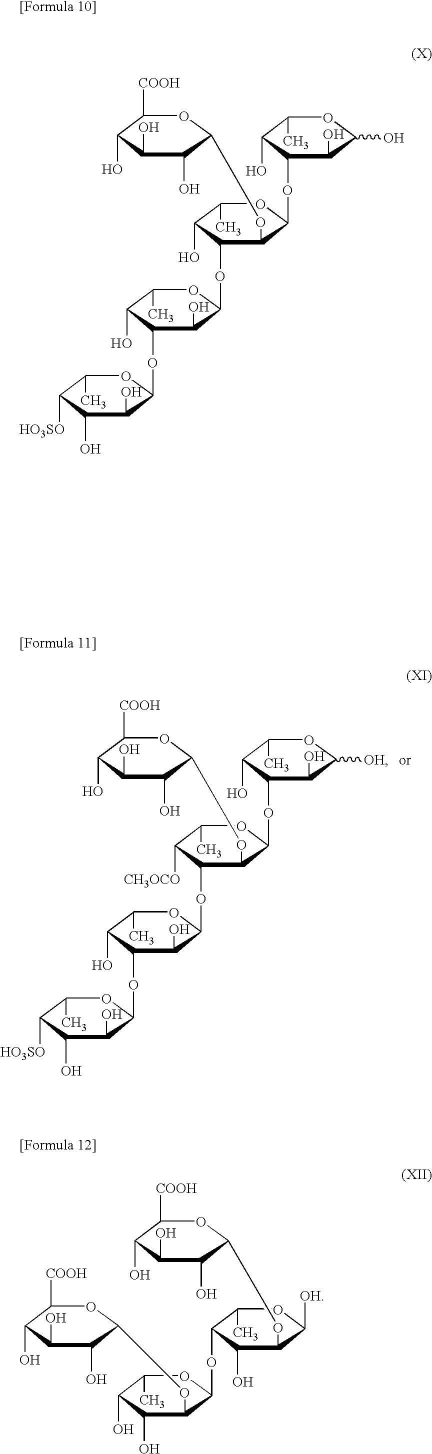

3. A lipase inhibitor or an α-glucosidase inhibitor, which comprises at least one member selected from the compounds represented by formulae (I) to (XII) as defined in claim 1.

4. An anti-obesity or blood glucose elevation suppressing agent, which comprises at least one member selected from the compounds represented by formulae (I) to (XII) as defined in claim 1.

5. An anti-obesity or blood glucose elevation suppressing agent based on inhibition of carbohydrate absorption through α-glucosidase inhibition, which comprises at least one member selected from the compounds represented by formulae (I) to (XII) as defined in claim 1.

6. An anti-obesity agent based on lipase inhibition, which comprises at least one member selected from the compounds represented by formulae (I) to (XII) as defined in claim 1.

7. An anti-obesity agent having both lipase inhibition activity and α-glucosidase inhibition activity, which comprises at least one member selected from the compounds represented by formulae (I) to (XII) as defined in claim 1.

8. A food or beverage, which incorporates at least one member selected from the compounds represented by formulae (I) to (XII) as defined in claim 1.

9. A pharmaceutical composition, which comprises at least one member selected from the compounds represented by formulae (I) to (XII) as defined in claim 1.

10. A cosmetic, which comprises at least one member selected from the compounds represented by formulae (I) to (XII) as defined in claim 1.

11. A lipase inhibitor or an α-glucosidase inhibitor, which comprises a composition obtained by hydrolysis of fucoidan with 0.1 to 5 N acid at 25° C. to 130° C. for 15 minutes to 6 hours.

12. The lipase inhibitor or α-glucosidase inhibitor as defined in claim 11, which comprises a composition obtained by hydrolysis of fucoidan with 1 to 2 N acid at 50° C. to 105° C. for 15 minutes to 3 hours.
